(19) United States
(12) Patent Application Publication
(10) Pub. No.: US 2008/0283049 A1
(43) Pub. Date: Nov. 20, 2008

(54) HIGH EFFICIENCY NEBULIZER

(76) Inventors: Derek D Mahoney, Manalapan, NJ (US); George V. Muttathil, Cherry Hill, NJ (US); Kerry D. O'Mara, Hopewell Twp., NJ (US); Albert F. Stevens, Moorestown, NJ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/935,472

(22) Filed: Nov. 6, 2007

Related U.S. Application Data
(60) Provisional application No. 60/891,892, filed on Feb. 27, 2007.

(51) Int. Cl. A61M 11/06

(52) U.S. Cl. 128/200.14

(57) ABSTRACT

The present invention relates generally to a nebulizer, and more particularly but not exclusively to a compact nebulizer that efficiently utilizes medication.

### RELATED APPLICATIONS

This application claims the benefit of priority of U.S. Provisional Applications Nos. 60/891,892 filed on Feb. 27, 2007 and 60/999,755 filed on Aug. 9, 2007, the entire contents of which application are incorporated herein by reference.

### FIELD OF THE INVENTION

The present invention relates generally to a nebulizer, and more particularly but not exclusively to a compact nebulizer that efficiently utilizes medication.

### BACKGROUND OF THE INVENTION

The deposition efficiency in the tracheobronchial (TB) and pulmonary regions is highly dependent on particle size. Particle sizes in the range of about 1 to 5 μm, as well as the size range extending from approximately 0.005 to 0.5 μm, have a relatively high rate of deposition within the aforementioned regions. (See William Hinds, Aerosol Technology, p 241 (1999).) Various methods have typically been used to generate these therapeutic fine particles, such as air-blast nebulizers (i.e., compressed air, jet, or venturi nebulizer), pressure nebulizers, ultrasonic nebulizers, a vibrating orifice, a spinning disk, condensation devices, and inkjet technology-based nebulizers. However, despite the variety of methods used to generate therapeutic fine particles, problems remain such as wasted medication that is not dispensed and the swallowing of liquid medication by the user. Currently available nebulizers typically have residual (i.e., waste) medication of 50% or more. This waste is largely due to the fact that existing nebulizers will generate and disperse large and small particles. The large particle dispersion is not well controlled and leads to residual medication in the nebulizer and associated apparatus. Additionally, some nebulizers are relatively bulky, which unfortunately provides considerable surface area for medication deposition within the device which in turn leads to wasted unused medication. Thus, it would be an advance in the state of nebulizer art to more efficiently dispense and utilize liquid medication to reduce waste and increase patient compliance, and to protect the user of the nebulizer from swallowing liquid medication.

### SUMMARY OF THE INVENTION

In one of its aspects, the present invention provides a nebulizer comprising an impactor having a curved surface and a nozzle oriented so that outflow from the nozzle engages the curved surface. The nebulizer incorporates a nebulizer tube, which may comprise a single-piece, and that may include a convergent-divergent air mixing nozzle, as well as an integral feed channel for siphoning medication. The nebulizer tube independently provides a first-level (i.e., relatively coarse) nebulization. To obtain the fine particles desired for nebulizers, the output stream from the nebulizer tube is directed towards an impactor having a curved surface at, or proximate, the impact site. When the flow strikes the impactor, very fine particles are generated. The curvature of the impactor promotes two very desirable effects. First, the portion of the flow that is not atomized into very fine particles will drain down the impactor and return to a medication reservoir disposed under the impactor, creating a “waterfall” recycling effect. Second, the impactor curvature also helps to direct the nebulized medication in a preferred direction, in this case toward the user's mouth.

In another of its aspects, the present invention also reduces the risk to the user associated with the inadvertent swallowing of unacceptably large quantities of liquid medication present in the nebulizer's reservoir. This could occur if the patient were to tilt his or her head too far back. To substantially reduce this risk, a semi-permeable membrane or other suitable material that is permeable to mist but sufficiently impermeable to liquid may be deployed to allow delivery of the nebulized mist to the user but prevent the flow of bulk liquid medication.

The present invention also provides in one of its aspects a reduction in the necessary treatment time through the generation of a dense mist of particles, in part because the particles are in the correct size range for effective deposition in the desired TB or pulmonary regions. The relatively higher density of nebulized particles may be created with the use of multiple jet impactors. Within a single nebulizer assembly, two, three, or more, high velocity jets of liquid-carrying gas may be directed at an impactor surface, creating a relatively higher density of fine droplets. Thus, the patient can inhale the full dose of medicine in a shorter time from which three benefits follow: more rapid treatment in critical situations, a financial benefit for the clinical setting (i.e., less time required from medical staff), and higher patient compliance in the home setting.

In these regards, the present invention provides a nebulizer for delivering a mist of liquid, comprising a housing and a reservoir disposed internally to the housing for containing liquid to be nebulized by the nebulizer. The nebulizer may include a monolithic nebulizer tube which has a gas channel having a first end for receiving a gas, such as compressed gas, and a second end for expelling the compressed gas and/or liquid. The gas channel may extend from a first end to a second end of the nebulizer tube. The monolithic nebulizer tube may also include a liquid feed channel comprising a first end in fluid communication with the reservoir for receiving liquid from the reservoir. Depending on the application the liquid may desirably be a liquid medication. The feed channel may include a second end in fluid communication with the gas channel. Alternatively, the feed channel may have an annular passageway at a second end of the feed channel with the annular passageway disposed about the second gas channel end. Application of compressed gas to the first end of the gas channel creates a siphon in the liquid feed channel to draw liquid into the feed channel and to expel the liquid and compressed gas from the second end of the nebulizer tube. To direct the flow of nebulized mist to an exit port of the nebulizer, a tortuous passageway may be provided between the second end of the gas channel and an exit port of the nebulizer. The tortuous passageway may be configured to remove nebulized particles larger than a selected therapeutic size from the flow of nebulized mist.

The nebulizer may further include an impactor disposed proximate the second end of the gas channel to nebulize the expelled liquid when the expelled liquid strikes the impactor. The impactor may be disposed sufficiently close the second end of the gas channel to assist in nebulizing the liquid expelled from the second end of the gas channel. The impactor may comprises a spherical, cylindrical, or mesa-like shape, or may include a ring disposed around the mesa to provide an annular channel between the ring and the mesa. The annular channel may be dimensioned to provide a fundamental resonant frequency of the annular channel tuned to generate particles of a preferred size.

In another configuration, the present invention provides a nebulizer for delivering a mist of liquid, comprising a housing having an inlet port for receiving compressed gas, such as compressed air for example, and an exit port for delivering a mist of nebulized liquid. A reservoir is disposed internally to the housing for containing liquid to be nebulized by the nebulizer. The nebulizer also includes a nebulizer tube in fluid communication with the liquid having an outlet from which the nebulized mist is provided. The outlet end of the nebulizer tube is disposed internally to the housing. The nebulizer also includes a tortuous passageway disposed within the housing between outlet end of the nebulizer tube and the exit port of the nebulizer for directing the flow of nebulized mist therethrough to the exit port.

In yet another configuration, the present invention provides a nebulizer for delivering a mist of liquid, comprising a two-piece housing having separate first and second housing portions, and a reservoir monolithic to the housing for containing liquid to be nebulized by the nebulizer. The nebulizer includes a nebulizer tube monolithic to the housing. The nebulizer tube includes a gas channel having a first end for receiving a gas, such as compressed air for example, and a second for expelling compressed gas and liquid. The gas channel extends from the first end to a second end of the nebulizer tube. The nebulizer tube also includes a liquid feed channel comprising a first end in fluid communication with the reservoir for receiving liquid from the reservoir and comprising a second end in fluid communication with the gas channel. Application of compressed gas to the first end of the gas channel creates a siphon in the liquid feed channel to draw liquid into the feed channel and to expel the liquid along with compressed gas from the second end of the nebulizer tube.

### BRIEF DESCRIPTION OF THE DRAWINGS

The foregoing summary and the following detailed description of the preferred embodiments of the present invention will be best understood when read in conjunction with the appended drawings, in which:

FIG. 1 schematically illustrates a perspective view of a first exemplary nebulizer of the present invention;

FIG. 2 schematically illustrates the nebulizer of FIG. 1, but without the semi-permeable membrane in place;

FIG. 3 schematically illustrates a cross-sectional view of the nebulizer of FIG. 2 taken along the sectioning line 3-3;

FIGS. 4A and 4B schematically illustrate perspective views of exemplary configurations of the lower housing of a nebulizer;

FIGS. 5A and 5B schematically illustrate perspective views of exemplary configurations of the lower housing of a nebulizer of the present invention having an enlarged region for receiving liquid medication;

FIGS. 6, 7A, and 7B schematically illustrate perspective views of exemplary configurations of the upper housing of the nebulizer of the present invention;

FIG. 8 schematically illustrates a cross-sectional view of a nebulizer similar to that depicted in FIG. 3, but including the lower housing of FIG. 4B and the upper housing of FIG. 7A;

FIG. 9 schematically illustrates the cross-sectional view of the nebulizer of FIG. 3 with the lower housing removed and with the upper housing rotated to show the internal cavity facing upward;

FIG. 10 schematically illustrates the perspective view of the nebulizer of FIG. 2 with the lower housing removed and with the upper housing rotated to show the internal cavity facing upward;

FIGS. 11 and 12 schematically illustrate a perspective and cross-sectional view taken along the sectioning line 12-12, respectively, of a nebulizer tube of the present invention;

FIG. 13 schematically illustrates a perspective view of a second exemplary nebulizer of the present invention;

FIG. 14 schematically illustrates a cross-sectional view of the nebulizer of FIG. 13 taken along the sectioning line 14-14;

FIG. 15 schematically illustrates a perspective view of the lower housing of the nebulizer of FIG. 13;

FIG. 16 schematically illustrates a perspective view of the lower housing of the nebulizer of FIG. 13 with the nebulizer tube in place;

FIG. 17 schematically illustrates the nebulizer tube of FIG. 13 having a key for insertion in the upper housing;

FIG. 18 schematically illustrates a perspective view of the upper housing of the nebulizer of FIG. 13 having a keyway for receiving the key of the nebulizer tube;

FIG. 19 schematically illustrates a perspective view of the upper housing of the nebulizer of FIG. 13 with the nebulizer tube in place with the key of the nebulizer tube disposed in the keyway of the upper housing;

FIGS. 20A and 20B schematically illustrate perspective views of a liquid fill cap;

FIGS. 21A and 21B schematically illustrate alternative airfoil shapes for the impactor;

FIG. 22 schematically illustrates a perspective view of a third exemplary nebulizer of the present invention;

FIG. 23 schematically illustrates a cross-sectional view of the nebulizer of FIG. 22 taken along the sectioning line 23-23;

FIG. 24 schematically illustrates a perspective view of the lower housing of the nebulizer of FIG. 22;

FIGS. 25, 26, and 27 schematically illustrate perspective views of the upper housing of the nebulizer of FIG. 22;

FIG. 28 schematically illustrates a cross-sectional view of the upper housing of FIG. 26 taken along the sectioning line 28-28, having a three-channel nebulizer tube in place of the single channel nebulizer tube of FIG. 22;

FIG. 29 schematically illustrates a cross-sectional view of the upper housing of the nebulizer of FIG. 26 taken along the sectioning line 29-29;

FIGS. 30 and 31 schematically illustrate a perspective and cross-sectional view taken along the sectioning line 31-31, respectively, of a nebulizer tube of the present invention having three outlet channels;

FIGS. 32 and 33 schematically illustrate a perspective and cross-sectional view taken along the sectioning line 33-33, respectively, of a nebulizer tube of the present invention having two outlet channels;

FIGS. 34 and 35 schematically illustrate a perspective and cross-sectional view taken along the sectioning line 35-35, respectively, of a nebulizer tube of the present invention having one outlet channel;

FIG. 36 schematically illustrates a perspective view of a lower housing, similar to the housing shown in FIG. 24, but having make-up air curtain walls;

FIG. 37 schematically illustrates a perspective view of an upper housing, similar to the housing shown in FIG. 25, but having make-up air curtain walls;

FIG. 38 schematically illustrates a perspective view of a fourth exemplary nebulizer of the present invention having two parts with an monolithically integrated nebulizer tube;

FIG. 39 schematically illustrates a perspective view of the nebulizer of FIG. 38 with the lid open;

FIG. 40 schematically illustrates a perspective view of the nebulizer similar to that shown in FIG. 39 but having a two channel nebulizer tube;

FIG. 41 schematically illustrates a cross-sectional view taken along the sectioning line 41-41 of the nebulizer of FIG. 38;

FIG. 42 schematically illustrates a cross-sectional view taken along the sectioning line 42-42 of the nebulizer of FIG. 38;

FIG. 43 schematically illustrates a perspective view of the upper housing of the nebulizer of FIG. 38;

FIG. 44 schematically illustrates a cross-sectional view of the upper housing taken along the sectioning line 44-44 of FIG. 43 with the top cut away;

FIG. 45 schematically illustrates a perspective view of the lower housing of the nebulizer of FIG. 38;

FIG. 46 schematically illustrates a cross-sectional view taken along the sectioning line 46-46 of the lower housing of FIG. 45;

FIGS. 47 and 48 schematically illustrate a perspective and cross-sectional view taken along the sectioning line 48-48, respectively, of a nebulizer tube of the present invention having an annular medication delivery port;

FIGS. 49 and 50 schematically illustrate a perspective and cross-sectional view taken along the sectioning line 50-50, respectively, of an upper housing having a spherical impactor;

FIG. 51 schematically illustrates a fragmentary cross-sectional view of the upper housing of FIG. 49 and a lower housing assembled with the nebulizer tube of FIG. 47 disposed therein;

FIGS. 52 and 53 schematically illustrate a perspective and cross-sectional view taken along the sectioning line 53-53, respectively, of an upper housing having a cylindrical impactor;

FIGS. 54 and 55 schematically illustrate a perspective and cross-sectional view taken along the sectioning line 55-55, respectively, of an upper housing having a mesa-shaped impactor; and

FIGS. 56 and 57 schematically illustrate a perspective and cross-sectional view taken along the sectioning line 57-57, respectively, of an upper housing having a mesa-shaped impactor with a ring disposed about the mesa to provide a resonant annular channel.

### DETAILED DESCRIPTION OF THE INVENTION

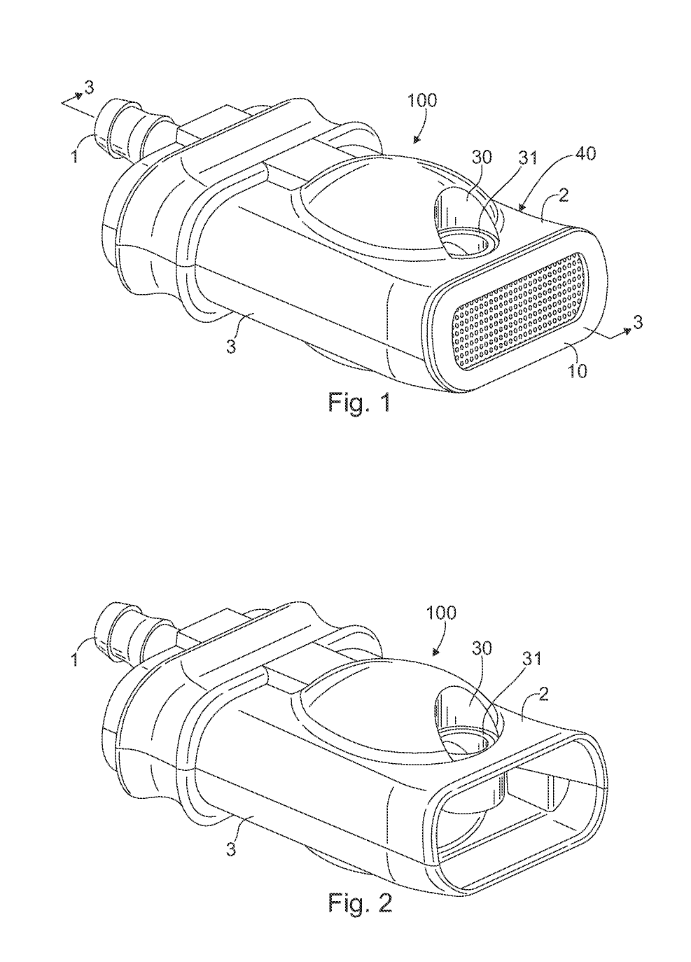

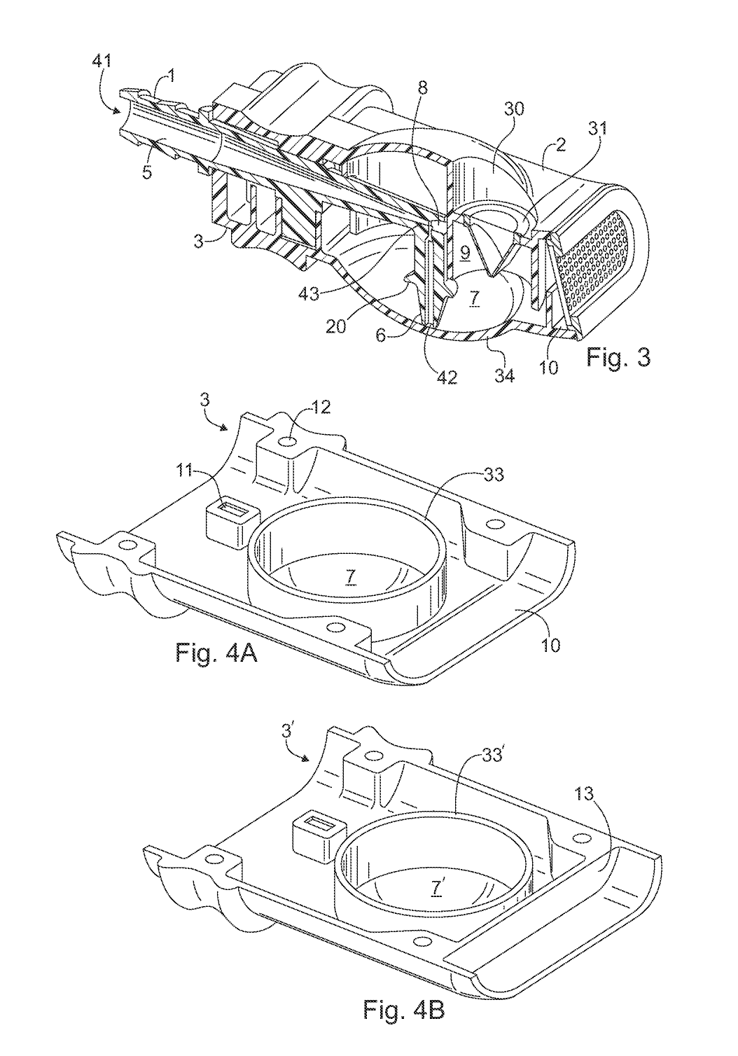

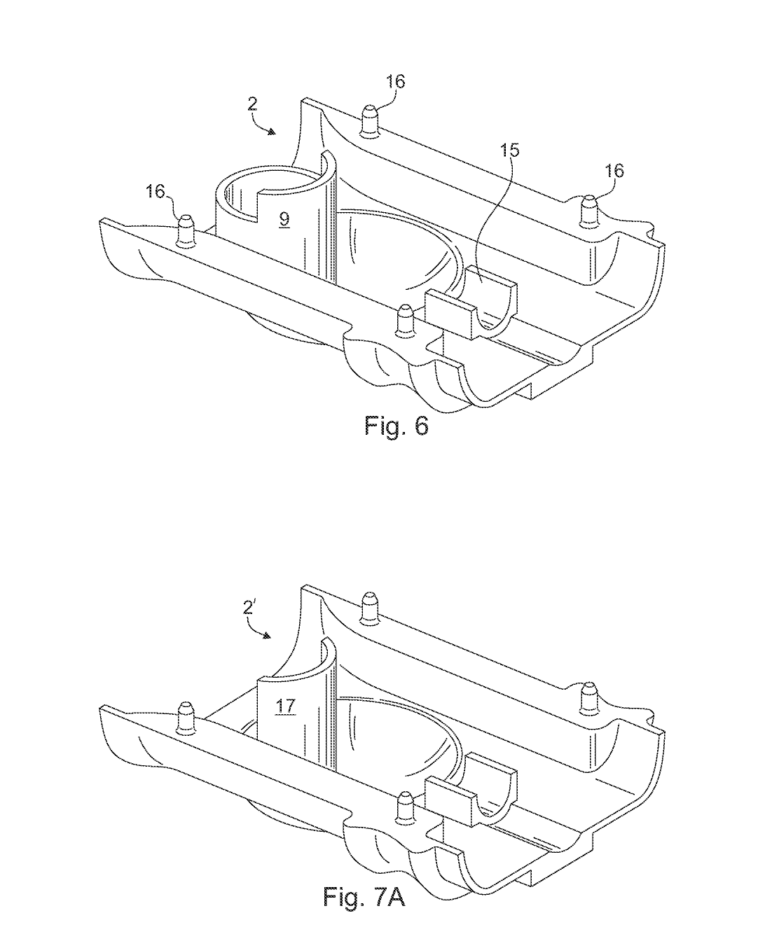

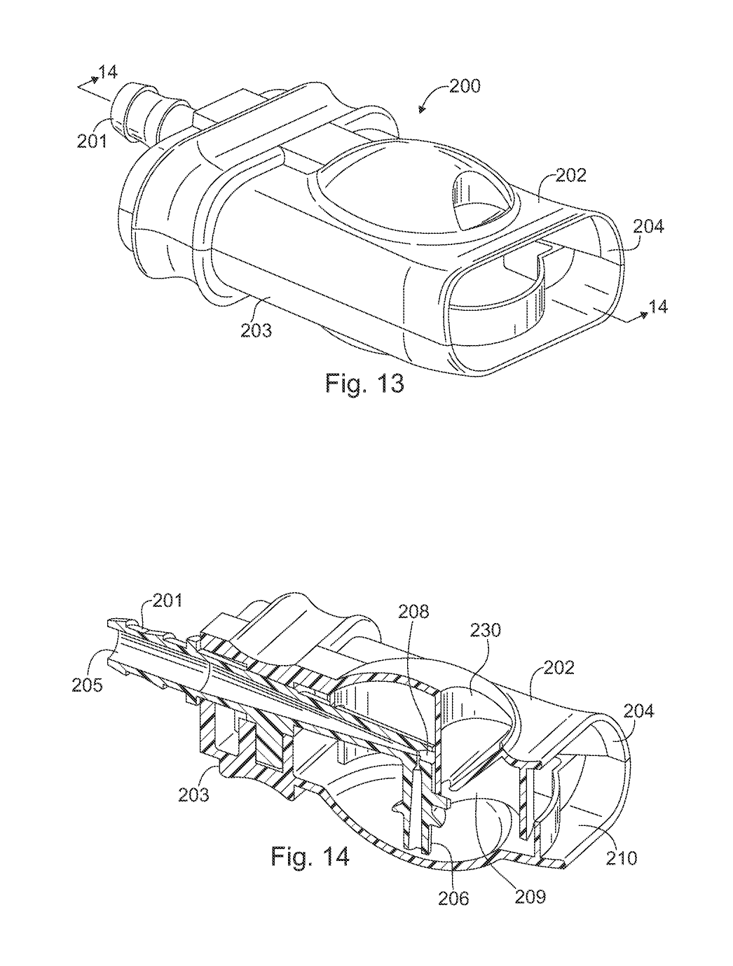

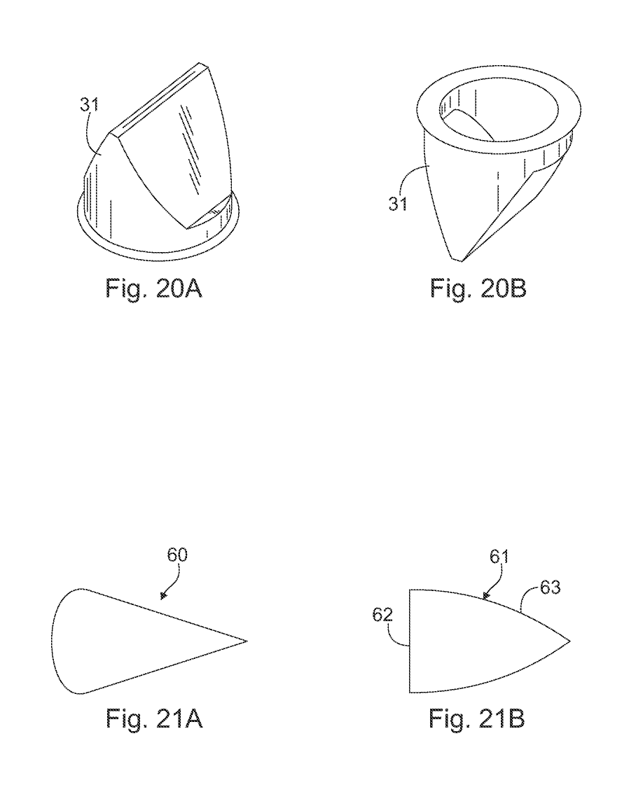

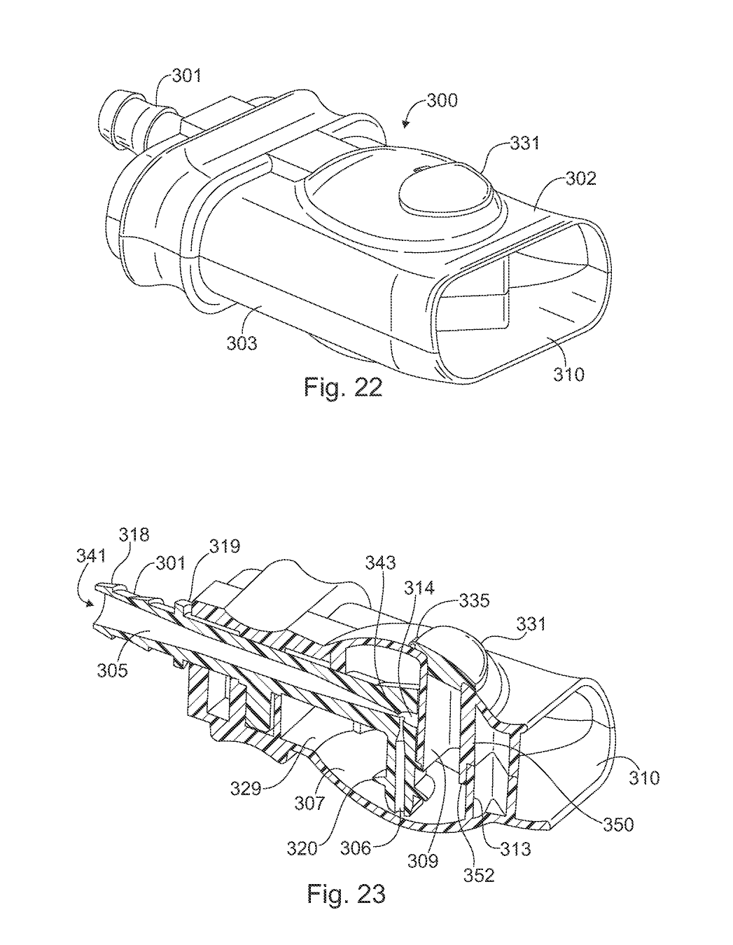

Referring now to the figures, wherein like elements are numbered alike throughout, FIGS. 1 and 2 illustrate an external view of a first configuration of a nebulizer 100 of the present invention. The nebulizer 100 comprises a nebulizer tube 1 disposed within a housing 40 for receiving compressed gas, such as compressed air or nitrogen, for example, and an exit port 10 for delivering a nebulized mist to a user. The housing 40 may comprise an upper housing 2 and a lower housing 3, which may be registered to one another by cooperation between holes 12 of the lower housing 3 and alignment posts 16 of the upper housing 2, FIGS. 4A, 6. The upper housing 2 may include a fill port 30 for introducing a liquid medication into the housing 40. The fill port 30 may be shaped to readily accept the shape of standard medicine containers, which will facilitate filling of the nebulizer 100 with the correct amount of medication and reduce the possibility of spillage and waste. The fill port 30 may remain open and may also serve as an exit for nebulized liquid, or the fill port 30 may optionally include a separate funnel or duckbill-shaped cap 31 for insertion into the upper housing 2 to direct the liquid medication into the housing 40, FIGS. 1, 3, 20A, 20B. The cap may be located inside the main body of the nebulizer to deter the cap from inadvertently coming loose and being swallowed by the user. Alternatively, the fill port cap 230 may be provided as an integral portion of the upper housing 202, FIGS. 13, 14. The cap 31, 230 may be configured so that it deflects to permit liquid to be poured into the nebulizer when a small force applied. For example, the cap 31, 230 may deflect when a syringe is inserted for delivering liquid and may close again after the syringe is removed, or the cap 331 may be molded as part of the upper housing 302 and connected thereto via a living hinge 335, FIGS. 22 and 23. Moreover, the cap 31 may be provided in the form of a one-way duck-bill valve that permits the entry of liquid medication but deters the flow of nebulized mist therethrough.

To receive a liquid, such as medication, introduced through the fill port 30, the lower housing 3 includes a reservoir 7 which may include a cylindrical sidewall 33 for containing the liquid medication within a localized region within the lower housing 3. (While any suitable liquid may be provided in the reservoir, for illustration purposes the devices of the present application are described herein as containing a medication.)

The reservoir 7 may be dimensioned to hold at least 3 ml of liquid medication, for example. In addition, to further contain the location of the liquid medication, the reservoir 7 may include a hemispherical or other suitably shaped depression 34 into which the liquid medication may pool. Maintaining the liquid medication in a specified location assists in making the medication available to the nebulizer tube 1, and thus aids in efficient use of the medication.

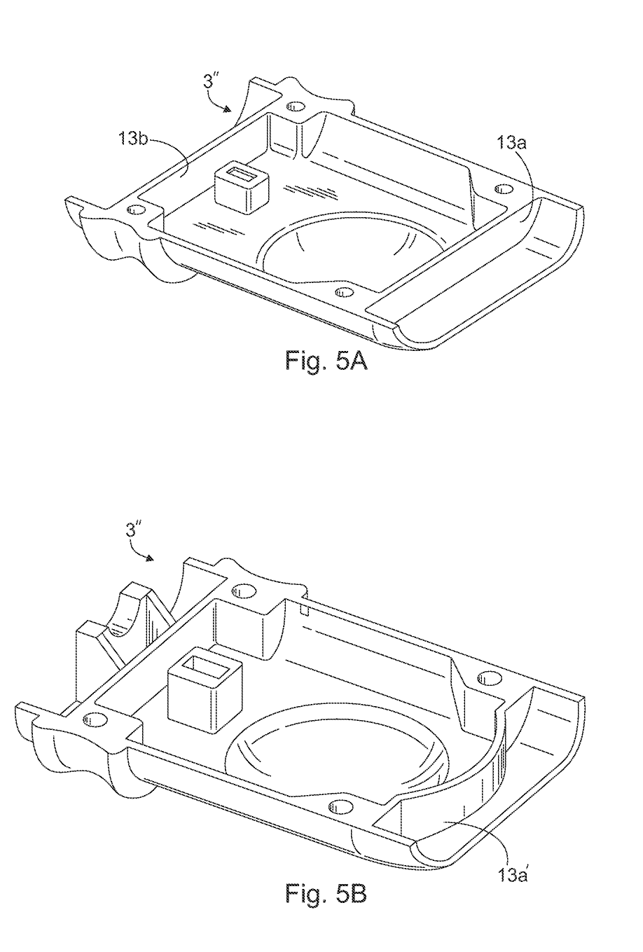

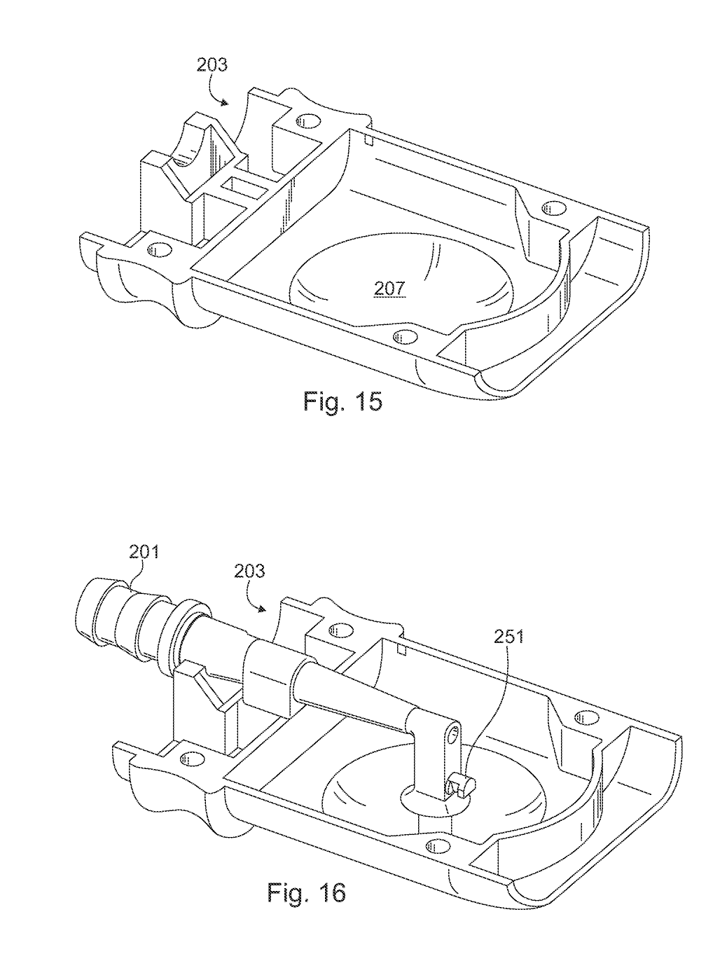

The reservoir 7 may include shapes other than cylindrical. For example, the reservoir 7″ may have a generally rectangular shape being bounded at the inlet and outlet end of the lower housing 3″ by front and rear reservoir walls 13a, 13b, FIG. 5A. The reservoir walls 13a, 13b may be straight, curved 13a′, or assume any other suitable shape, FIGS. 5B, 16. In addition, in the event that liquid medication overflows the wall 33′ of the reservoir 7′, an overflow wall 13 may optionally be provided at the exit port 10 to help deter introduction of liquid medication into the user's mouth, FIG. 4B. Furthermore, one or more semi-permeable membranes 4 may be provided at the exit port 10 of the nebulizer 100 to permit mist flow while acting as an effective liquid barrier, thus creating a safety feature that prevents a user from swallowing liquid medication contained in the nebulizer 100. In one configuration the semi-permeable membranes 4 may be used instead of the front reservoir wall 13a. Alternatively, or additionally, an absorbent material, such as a sponge, may be incorporated into the nebulizer 100, for example between the reservoir 7′ and overflow wall 13, to deter the outflow of liquid medication into the exit port 10. For instance, in the event that the nebulizer is tilted beyond some critical angle during use, the membrane 4 and/or absorbent material will block the flow of medication into the user's mouth while permitting the nebulized mist to flow through the membrane 4.

For example, a foam sponge material may be used as the membrane 4 to permit mist flow while deterring liquid medication flow therethrough. In the nebulizers of the present invention, the flow of small droplets from the nebulizer 100 operates in a very low Reynold's number flow regime. The Reynold's number is a dimensionless number, a ratio of the momentum forces acting on a body to that of the viscous forces. In a low Reynold's number flow, particles tend to follow the path of the gas flow and are not likely to impact upon the solid surfaces that restrain the flow. This holds true even when that flow path is a circuitous one through the pores of a thickness of sponge material. The droplets are carried through with the flowing gas stream, and so the sponge remains dry.

Thus, in one embodiment of the present invention, the membrane 4 is provided in the form of a layer of sponge material that covers the flow through the exit port 10 and permits the nebulized mist to flow out. The sponge could comprise either a wettable or non-wettable material for the given liquid medication. (The determination of whether a material is “wetting” or “non-wetting” depends on the liquid being used. As used herein, we are most interested in the wettability of materials mainly as it pertains to the use of aqueous solutions.) If the sponge were non-wettable, a sufficiently small pore size would have enough capillary pressure to prevent the liquid medication from progress through the sponge membrane 4. (A simple, well known equation can be used to calculate the “capillary pressure.” Capillary pressure is the pressure that would be required to force the liquid through a given-sized circular hole in a non-wetting material. The capillary pressure is dependent upon: the contact angle, the surface tension of the liquid, and the diameter of the hole.) However, most readily available sponge materials are comprised of wettable materials. If a wettable sponge material were employed as the membrane 4, the wettable sponge material should be located so that it is not typically in contact with the bulk liquid medication in the nebulizer 100. Otherwise, the liquid medication would undesirably be wicked into the sponge and would not be available to be delivered to the user. Nonetheless, a wettable sponge can provide useful functionality when it is strategically located so that, if the nebulizer 100 is tilted too much, the sponge acts as a barrier wicking up the large liquid drops or liquid that has sloshed due to rapid gross motions of the nebulizer 100. For example, a 2 mm thick layer of polyethylene wettable foam having about 80% open space and pore sizes of about 0.8 mm may be used as the membrane 4. In addition, a foam layer in the flow exit path proximate the exit port 10 provides an additional feature: a very slight back-pressure in the flow path of the gas and liquid mixture (i.e. the airborne droplets). This slight back-pressure gives the effect of a diffuser by evening out the velocity profile at the nebulizer exit port 10 so that the nebulized mist exits the nebulizer 100 at a slower average velocity and more uniform distribution across the exit port 10. (The diffuser effect causes the velocity to be more uniform. The slight flow restriction or back pressure, due to the presence of the foam layer, will tend to slow the flow.)

Further exemplary materials for use as the membrane 4 would include films comprised of fluoropolymers (PTFE, etc.), such as DuPont Teflon® PTFE, having very small pore sizes. Films such as these are currently being produced by W. L. Gore Company under the Gore-Tex® trademark. Teflon® PTFE has a very low surface energy as it is essentially a non-polar molecule. Water is a polar molecule, and liquid water does not “wet” a Teflon® PTFE surface. Instead, liquid water forms “beaded” drops on the surface of the Teflon® PTFE; each drop has a contact angle much greater than 90 degrees. In the case of liquid water and Teflon® PTFE, a very high pressure is required to force water through small holes in the material. However, gases and water mist flow through the pores with little trouble. Gore-Tex® films are specifically created to exploit this phenomena in a number of applications. (One example is a “T” fitting that has one port covered by Gore-Tex® film. This assembly is used in some intravenous tubing, which allows gases to vent out of the tube but prevents the IV fluid from leaking through.)

The nebulizer tube 1 includes a liquid feed channel 6 having an inlet end 42 disposed in fluid communication with the reservoir 7 to receive liquid medication disposed within the lower housing 3, FIGS. 3, 12. The feed channel 6 communicates with a gas channel 5 of the nebulizer tube 1 to deliver the liquid medication to the gas channel 5 to be nebulized. The gas channel 5 includes an inlet end 41 for connection to a source of compressed air and a throat 43 where the feed channel 6 connects to the gas channel 5. The gas channel 5 may be provided in the form of a convergent channel 5 that has a cross-sectional dimension that decreases from the inlet end 41 to the throat 43 where the cross-sectional dimension may be a minimum, e.g., 15 to 20 thousandths of an inch. The feed channel 6 may also have a minimum cross-sectional dimension at the throat 43, e.g., 15 to 20 thousandths of an inch. The nebulizer tube 1 also includes a nozzle 8 disposed in fluid communication with the throat 43 of the gas channel 5. The nozzle 8 includes a channel cross-sectional dimension that increases away from the throat 43 towards the outlet end 44 of the nebulizer tube 1.

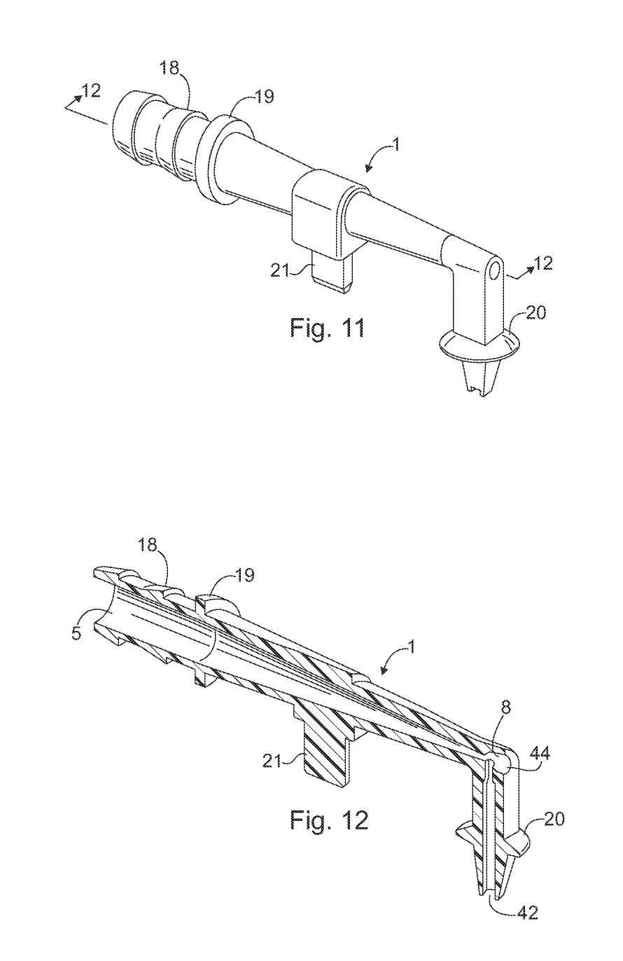

The inlet end 41 of the nebulizer tube 1 may include a barb 18 to assist in securing attachment of a compressed air hose to the inlet end 41 of the nebulizer tube 1, FIGS. 11, 12. A flange 19 may also be included to provide a positive stop for the air hose during initial installation. During operation, compressed air, of 25 to 45 psi for example, enters the convergent channel 5 of the nebulizer tube 1. The air accelerates until it reaches the throat 43 of the convergent channel 5. By virtue of the Bernoulli effect, as the flow velocity increases, its static pressure will decrease. As a result, the static pressure at the throat 43 of the convergent channel 5 will be below that of the local atmospheric pressure. Since the static pressure of the liquid is higher than the static pressure at the throat 43 of the nebulizer tube 1, liquid is siphoned upward into the feed channel 6 as a result of a venturi effect. Subsequent to siphoning, the liquid/air mixture is rapidly expanded in the divergent section of the nozzle 8. This rapid expansion encourages turbulent mixing and creates an effective first-level of nebulization.

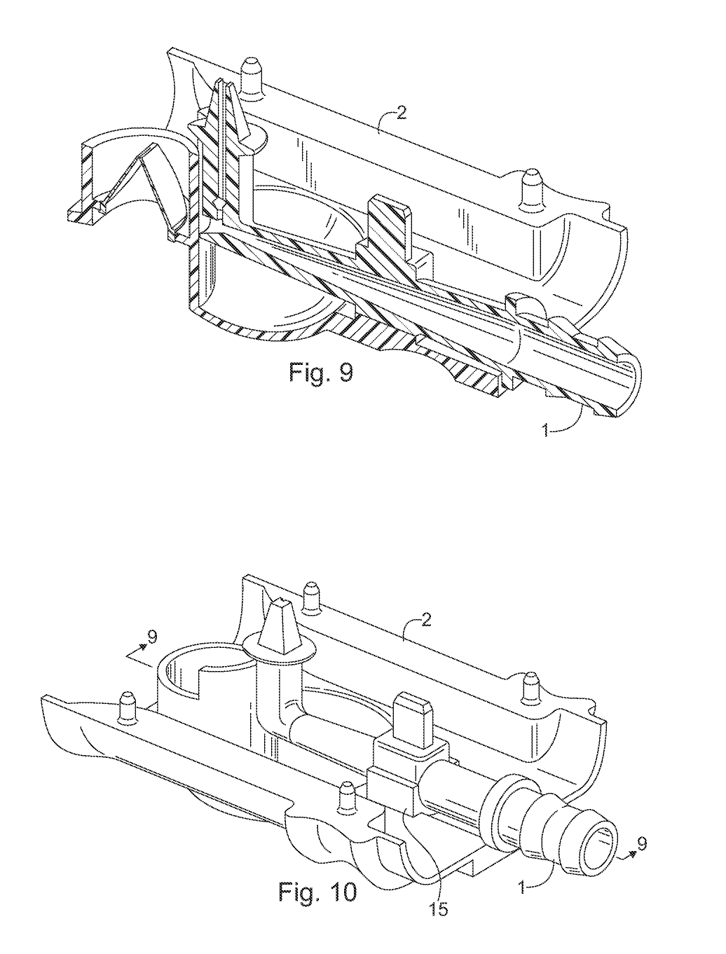

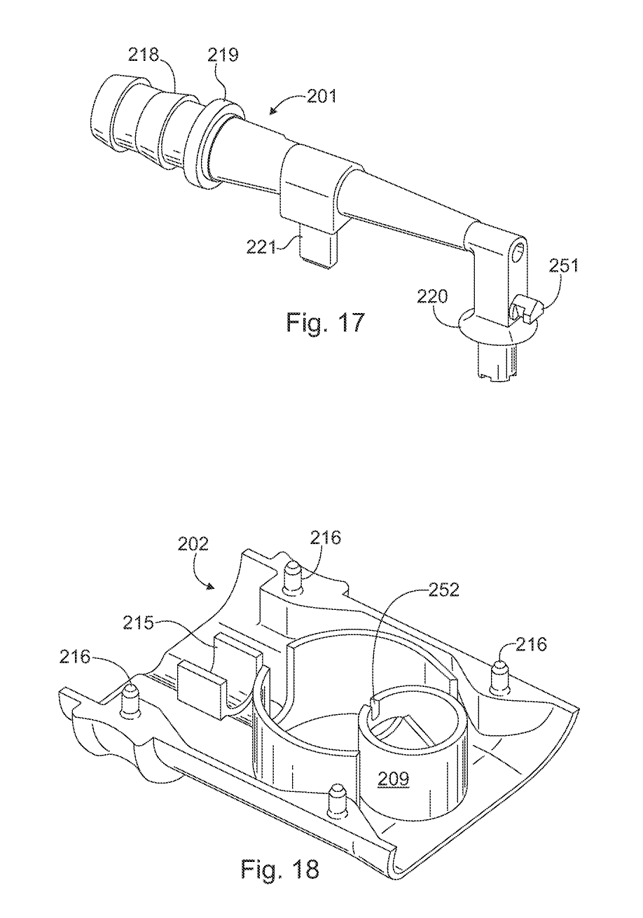

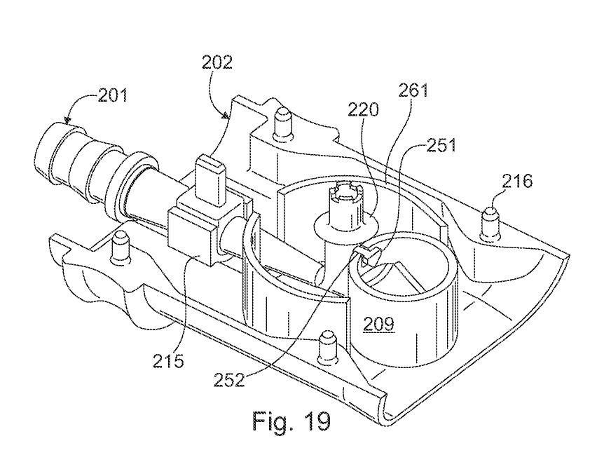

The nozzle 8 is oriented so that the output flow from the nozzle 8 strikes a curved impactor 9, which may be provided as part of the upper housing 2. This energetic collision generates the very fine, therapeutic particles required of nebulizers. It has been determined that a sufficiently small spacing is required between the nozzle 8 and impactor 9 to generate a fine mist. A suitable nozzle to impactor spacing is 10 to 20 thousandths of an inch. The location of the nozzle 8 relative to the curved impactor 9 may be specified by an alignment boss 21 provided on the nebulizer tube 1 that mates with a complementary positioning feature 11 of the lower housing 3 to locate the nebulizer tube 1 within the housing 40. In addition, the nebulizer tube mates with an nozzle capture feature 15 of the upper housing 2 to stabilize the tube 1 within the nebulizer 100, FIGS. 8-10. Additionally, or alternatively, registration of the nebulizer tube 1 to the impactor 9 may be provided by direct or indirect physical cooperation between the nebulizer tube 1 and impactor 9. For example, referring to FIGS. 16-19 (wherein structures similar to those illustrated in FIGS. 1-12 are similarly numbered with a “200”-series reference numeral), the nebulizer tube 201 may include a registration feature, such as a boss or key 251, for mating with a complementary structure, such as keyway 252, on the nebulizer 209. Engagement between the key 251 and the keyway 252 establishes the relative position between the nozzle 208 and the impactor 209.

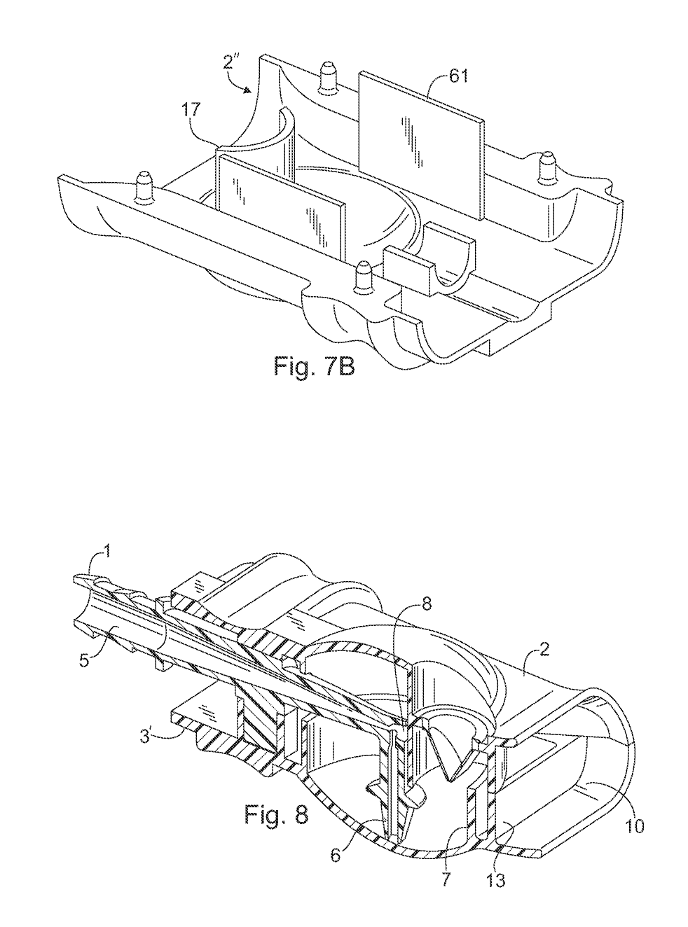

The impactor 9, 209 may have a generally cylindrical shape, such as a substantially full cylinder, FIG. 6, or a partial cylindrical impactor 17, FIG. 7A. Such impactor shapes will generate a fine mist and will also facilitate the flow of mist toward the user's mouth. Other curved surfaces may be substituted for the cylindrical impactors 9, 209 such as elliptical, or other suitable shape, e.g., an airfoil 60, FIG. 21A. In addition, the curved impactor may have a cross-sectional shape which includes a flat region 62 as well as a curved region 63, such as the airfoil 61 illustrated in FIG. 21B, for example. The airfoil impactor 60, 61 is oriented within the housing 40, 240 so that the tapered portion of the airfoil points in the downstream direction towards the exit port 10, 210 of the nebulizer 100, 200. Such an orientation of the airfoil impactor 60, 61 would reduce turbulence and backpressure of the air and mist as it moves out the exit port 10, 210 of the nebulizer 100, 200.

In addition to creating a fine mist, the curved impactor 9 also provides at least two other desirable functions: (I) it helps direct the nebulized mist towards the user's mouth, and (ii) it facilitates a waterfall-like recycling effect. The waterfall effect arises because part of the mixture exiting the nebulizer tube 1 will strike the impactor 9 and simply drain back down into the region containing the pool of liquid, i.e., reservoir 7. In this regard, the impactor 9 may be positioned above the reservoir 7. Of course, a significant portion of the air/liquid mixture will exit via port 10 of the nebulizer as a mist directed toward the user's mouth. An air baffle 20 may be provided on the nebulizer tube 1 proximate the feed channel inlet end 42, so that the high-velocity mixture striking the impactor 9 does not blow liquid away from the feed channel inlet 42 which could lead to a feed channel starvation condition. In addition, inclusion of the air baffle 20 can deter unwanted formation of large airborne droplets that might result from the surface of the liquid being agitated.

Additionally, the impactor 9, 209 can be shaped to create a scavenging flow within the nebulizer 100, 200. The scavenging flow would be directed throughout the housing interior and would help prevent the accumulation of medication on the internal walls of the nebulizer 100, 200. In addition, curtain walls 261 may be provided in the upper housing 2, 202 to redirect any accumulation of liquid on the upper surface of the upper housing 2, 202 downward into the reservoir 7, 207. The presence of curtain walls 261 can avoid the situation of liquid running down the interior sidewall of the upper housing 2, 202 to encounter and potentially leak out through the seam between the upper housing 2, 202 and the lower housing 3, 203. The curtain walls 261 may also be positioned sufficiently close to the impactor 209 to permit fine particles to travel around the impactor 209 to the exit port 210 and to cause larger particles to strike the curtain walls 261 and then drip down into the reservoir 207. Additionally, a filter-type material may be positioned in the nebulizer 100, 200 to give a preferential flow direction for the nebulized mist toward the user's mouth without creating an excessive flow resistance to inhalation. Furthermore, the housing 40, 240 and/or other components of the nebulizer 100, 200 may be fabricated from materials that possess surface tension properties characteristic of wetting materials to create a sheeting action that will facilitate the flow of recycled materials to the reservoir 7, 207. For example, the material of the housing 40 may comprise plastics that are non-wetting in their original condition. Polyethylene (PE) and polypropylene (PP) are two examples. If the reservoir 7 is constructed of one of these materials, and has sufficiently steep internal shape, the liquid medication will roll down to the lowest point, which would presumably be the location from which the liquid medication is being siphoned. Many times however, in practical applications, after having been used, a surface that started out as non-wetting, can become fully or partially wetting due to the deposition of a very thin layer of dirt, minerals, or other contaminants on the surface. The surface might then act as a wettable one. For this reason, it is important to design the reservoir 7 to work well as a wettable material to start with.

The wetting angle of a wettable material is less than 90 degrees. The contact angle can be a very small angle as the edge of a liquid is pulled along a solid surface. Several characteristics of a wettable surface, together with intentional geometric features, can be used to help the functionality of the nebulizer design. An ideal nebulizer would have the capability to utilize every bit of the liquid medication contained therein. Achievement of this goal may be attempted by pulling the liquid medication from a location that is the lowest point in a depression of the reservoir 7. The inner walls of the reservoir 7 may be sloped as much as possible, because as the liquid medication level goes down, droplets of water can remain stuck in random locations on the walls of a reservoir 7 that is made from a wettable material. These droplets would be counted as wasted medication that the nebulizer 100 is unable to use as residual content. The nebulizer design can cause the air flow to move generally downward along the walls of the reservoir 7, which is generally a turbulent action. However the shear action downward along the reservoir wall will scrub the liquid down toward the pick up location.

The geometry of the reservoir walls, together with the wetting characteristics of the reservoir can also help to reduce the amount of residual unused medication. Internal angles or grooves that run in a direction down the side walls of the reservoir 7 can also be included. The dimensions of the angles or grooves can be relatively small as compared with the dimensions of the reservoir 7, in which case the liquid will “wick” along the angles or grooves. Further, the design can be made to cause the liquid to preferentially move in one direction along the length of these features by gradually changing the size or shape of the groove along its length. For example, if the internal angle of the groove becomes more acute, the liquid will be preferentially pulled in that direction. Another technique for pulling the liquid toward the feed channel inlet 42 of the feed channel 6 is by make the gap between the bottom surface of the reservoir 7 and the feed channel inlet 42 sufficiently small to wick into this gap (if the surfaces are wetting materials). A further aid is to have the gap reduce in size (taper, or converge) as the liquid moves in the flow-wise direction, towards the feed channel inlet 42. A gap that becomes smaller as it approaches the inlet to the feed channel 42 can encourage the liquid to flow in that direction.

Turning next to FIG. 22, an additional configuration of a nebulizer 300 in accordance with the present invention is illustrated, in which the nebulizer is configured to reverse the flow of nebulized medication and then redirect the reversed flow towards the nebulizer exit port 310. The reversal and redirection of the flow of nebulized medication can serve as a particle size filter, allowing only the smaller sized particles to reach the nebulizer exit port 310. The three-piece nebulizer 300 includes a nebulizer tube 301, an upper housing 302, and a lower housing 303 along with an integral cap 331.

Referring to the cross-sectional view of FIG. 23, the structure of the nebulizer 300 and mechanism by which the nebulized mist is created may be understood. Compressed air enters the convergent gas channel 305 of the nebulizer tube 301 through an inlet end 341 of the nebulizer tube 301. A barb 318 may be incorporated into the nebulizer tube 301 to aid in securing an elastomeric air hose through which compressed air is introduced into the nebulizer tube 301. In addition, a flange 319 may be incorporated to provide a positive stop for the air hose during installation.

The air accelerates until it reaches the throat 343 (a location of minimum cross-sectional area) of the nebulizer tube 301. By virtue of the Bernoulli effect, as the flow velocity increases, its static pressure decreases. As a result, the static pressure at the throat 343 of the nebulizer tube 301 is below that of the local atmospheric pressure. An integral liquid feed channel 306 of the nebulizer tube 301 is disposed in communication with the medication located in the reservoir 307 of the lower housing 303. Since the static pressure of the liquid is higher than the static pressure at the throat 343 of the nebulizer tube 301, liquid is siphoned upward though the feed channel 306 as a result of this venturi effect. Subsequent to siphoning, the liquid/air mixture is rapidly expanded in the divergent section of the nozzle 314. This rapid expansion encourages turbulent mixing and creates an effective first-level of nebulization.

After exiting the nozzle 314, the mixture strikes an impactor 309 which may be provided as a monolithic part of the upper housing 302. This energetic collision generates very fine, therapeutic particles. The spacing between the nozzle 314 and the impactor 309 is selected to be sufficiently small, e.g., 20 to 40 thousandths of an inch, to generate a suitably fine mist. The impactor 309 also provides the waterfall-like recycling effect. An air baffle 320 of the nebulizer tube 301 is provided near the bottom of the feed channel 306 so that after the high-velocity mixture strikes the impactor 309 the deflected stream does not disturb the liquid near the feed channel inlet. Without the baffle 320, it is possible that a feed tube starvation condition could be created due to liquid being blown away from the feed channel 306. In addition, the surface of the liquid might be agitated to an extent that would produce unwanted formation of large airborne droplets. Note also, that in the event that the nebulizer is tilted forward beyond some critical angle during use, the adjoining walls 313, 350 of the upper and lower housings 302, 303 block the flow of medication into the user's mouth.

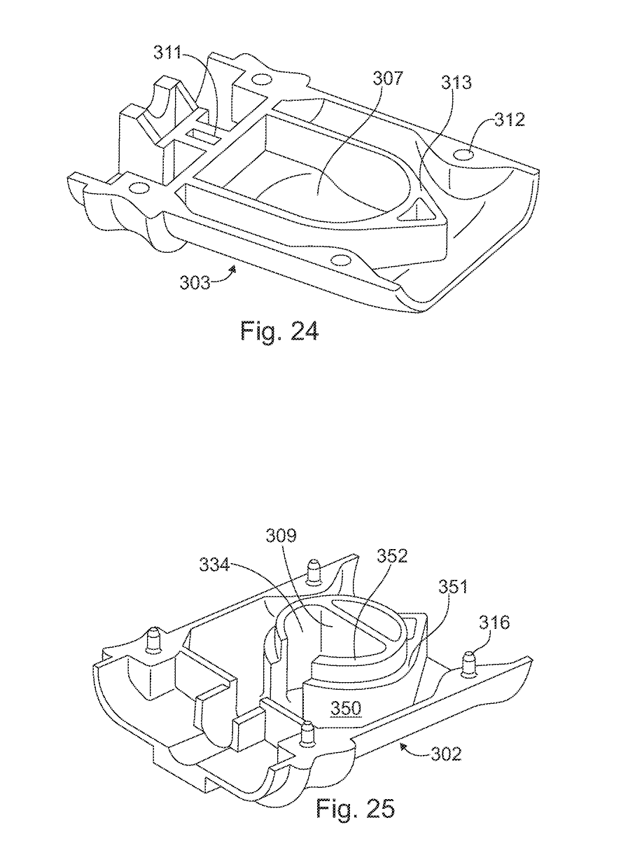

FIGS. 24-27 illustrate the lower and upper housings 302, 303 from which the structures that contribute to the reversal and redirection of the nebulizer flow through the nebulizer 300 can be seen. Turning first to the lower housing 303 of FIG. 24, the reservoir 307 may include a hemispherical or other suitably shaped depression for retaining liquid medication therein. The reservoir 307 may be surrounded by a reservoir wall 313, such as a U-shaped wall, that is configured to cooperate with corresponding structures in the upper housing 302 to aid in confining and directing the nebulized mist. (Additionally, an alignment feature 311 is provided to position the nebulizer tube 301 within the lower housing 303, and four holes (of which hole 312 is representative) are provided to align the upper and lower housings 302, 303 via the mating posts 316 of the upper housing 302, FIG. 25.)

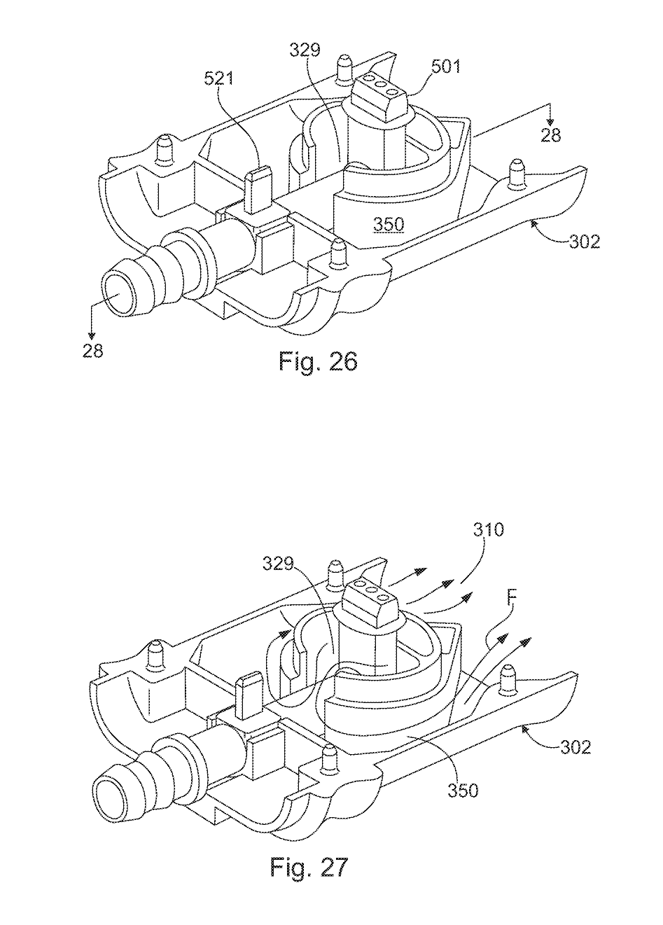

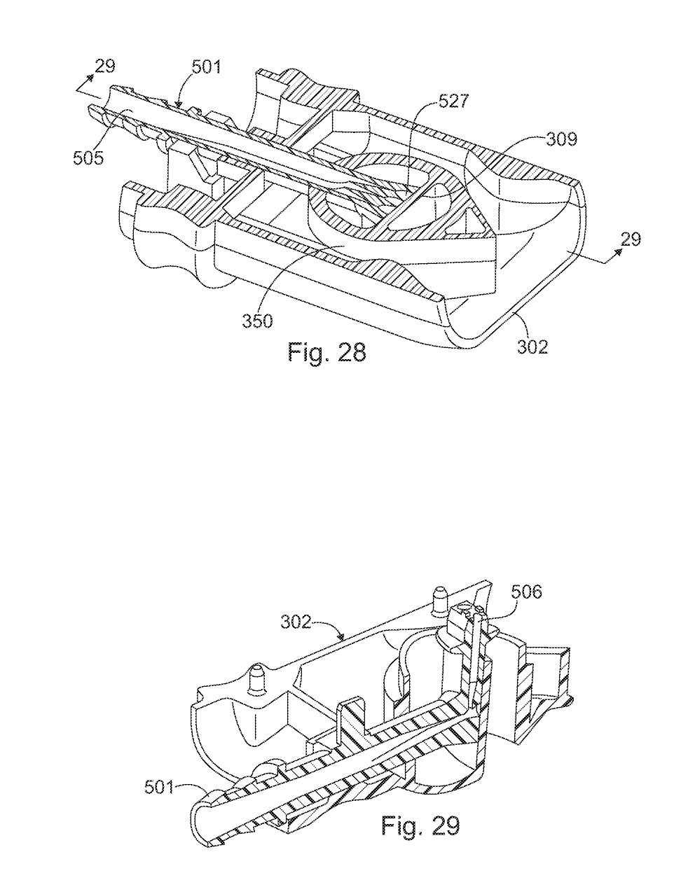

The upper housing 302 includes a nebulization chamber 334 in which the nebulized mist is generated, FIG. 25. The nebulization chamber 334 is defined by a chamber wall 350, which may have a generally cylindrical shape, and which optionally includes a shoulder 351 and an inset chamber wall portion 352 formatting with the lower housing 303 so that the shoulder 351 seats upon the upper surface of the reservoir wall 313 of the lower housing 303 and so that the inset chamber wall portion 352 extends into the cavity of the lower housing 303 defined by the reservoir wall 313, FIGS. 23-25. Also defining the nebulization chamber 334 is the impactor 309, which may be provided as a straight wall that spans the cylindrical space defined by the chamber wall 350. A chamber opening 329 is provided in the nebulization chamber wall 350 through which the nebulizer tube 301, 501 extends, FIGS. 23, 26. (As described more fully below the nebulizer tubes of the present invention can include multiple channels, such as the three-channel nebulizer tube 501 depicted in FIGS. 26-28.) As with the nebulizer configurations illustrated in FIGS. 1-19, the upper and lower housings 302, 303 may include analogous positioning features for registering the nebulizer tube 301, 501 relative to the upper and lower housings 302, 303, such as alignment boss 321 and complementary positioning feature 311, for example. The chamber opening 329 is dimensioned to be sufficiently large so that with the nebulizer tube 301, 501 in place a passageway is provided to allow the nebulized mist to exit the nebulization chamber 334 through the chamber opening 329. This geometry of the upper and lower housings 302, 303 is designed to provide a tortuous passageway to reverse and otherwise redirect the flow through the nebulizer 300, FIG. 27. In this regard, the tortuous passageway may comprise a first section for directing the flow, “F”, of nebulized medication away from the outlet end of the gas channel 305 at nozzle 314 and back towards the direction of the inlet end 341 of the gas channel 305 and may comprise a second section for directing the flow, “F”, of nebulized medication to the exit port 310 of the nebulizer 300.

Specifically, with reference to FIG. 27, the reverse flow geometry functions as follows. The mixture containing air and medication is directed through the nebulizer tube 301, 501 and exits the nebulizer tube 301, 501 striking the impactor 309. Since there is no immediate forward path toward the exit port 10 within the nebulization chamber 334, the nebulized mist is redirected out of the nebulization chamber 334 through the chamber opening 329 towards the rear of the nebulizer 300. By reversing the direction of the flow, particle size filtering occurs. Smaller particles that are able to quickly change direction will successfully exit the nebulization chamber 334. However, larger particles will impact upon the internal surface of the nebulization chamber 334 and will be recycled. The larger particles may then run down the internal surface of the chamber wall 350 to be deposited in the reservoir 307 so as to create a scavenging flow to minimize medication residuals. Upon exiting the chamber opening 329, since there is no exit port at the rear of the nebulizer 300, the flow of mist must again reverse direction in the direction of the exit port 310 to be emitted from the nebulizer 300. The redirection effectively serves as a particle size filter to ensure that therapeutic particles are emitted from the nebulizer 300. Additionally, to assist in diffusion of the nebulized flow as it exits the nebulizer 300, a taper 330 may be provided on the exterior of the nebulization chamber 334 in the form of an airfoil to diffuse the flow as the flow nears the exit port 310. The taper 330 also reduces the velocity, turbulence, and backpressure of the air and mist as it exits the nebulizer 300.

To further assist in directing airflow through the nebulizer to the patient, upper and lower housings 402, 403 may be provided which have a geometry that includes a flow path for external air to be drawn in by the patient, FIGS. 36, 37. In this regard, the upper and lower housings 402, 403 may be open at the end 412 opposite that of the exit port 410, and make-up air curtain walls 470, 474 may be included in the upper and lower housings 402, 403 provide make-up (or bypass) air passageways 472, 476 through the body of the housings 402, 403, allowing air to be drawn directly from the inlet end 412 through to the exit port 410.

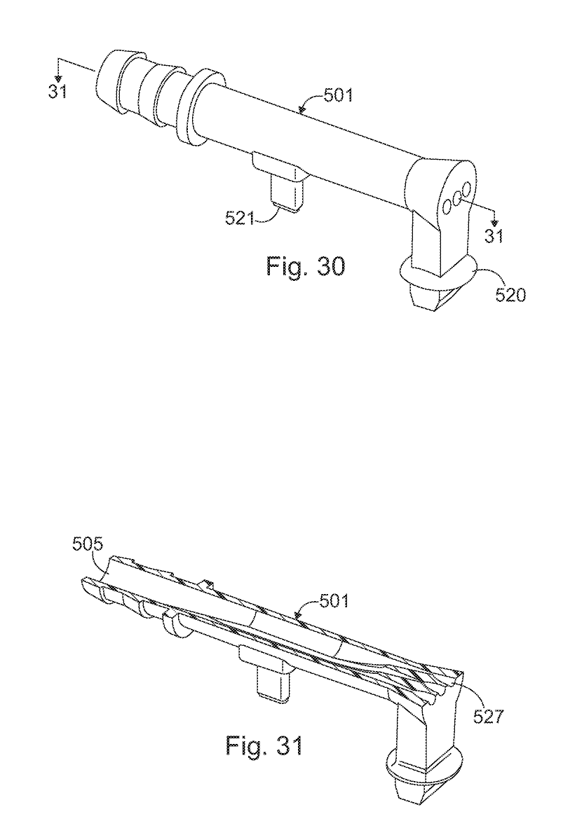

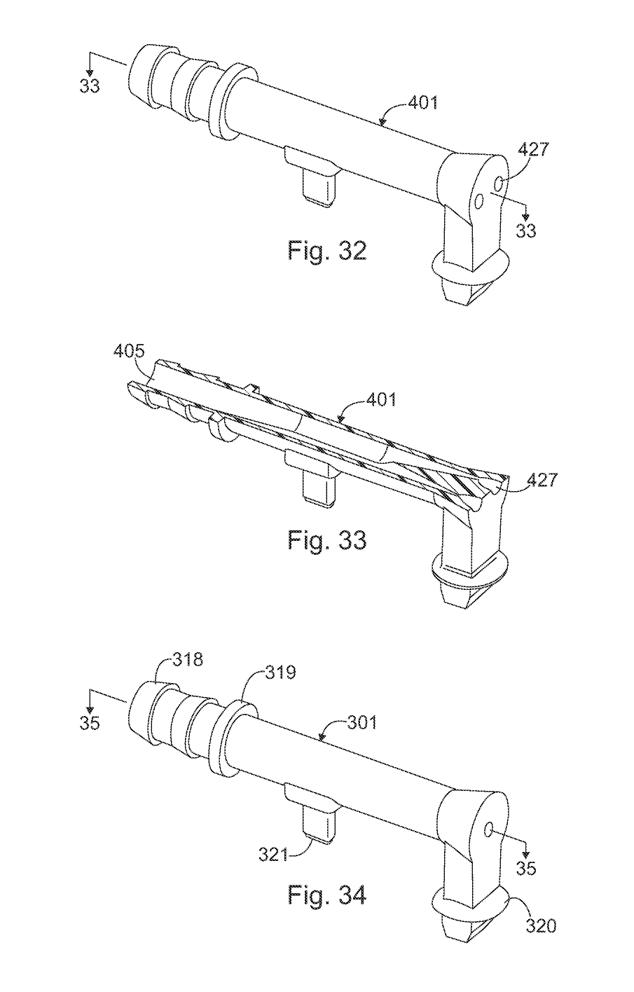

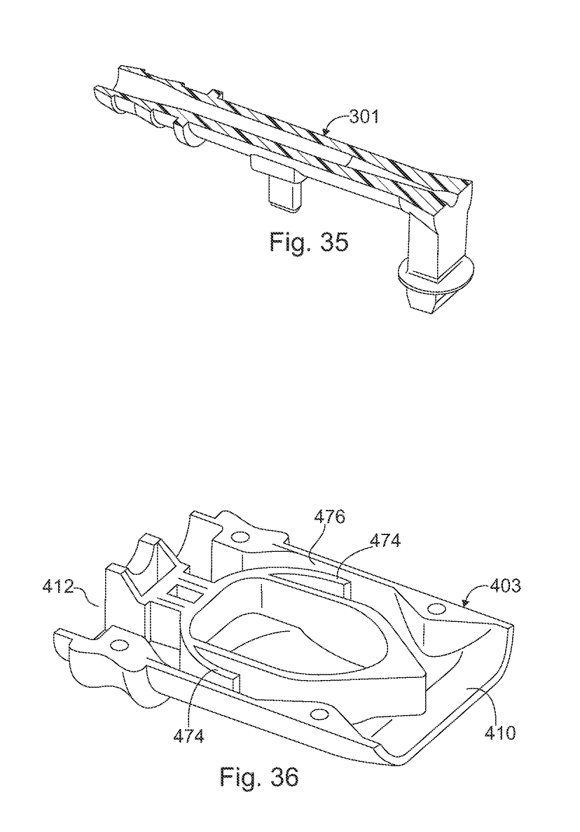

Each of the nebulizer configurations discussed so far may also utilize multi-channel nebulizer tubes 401, 501, rather than a single channel nebulizer tube 1, 201, 301, to reduce the treatment time. For example, as shown in FIGS. 26-33, the nebulizer 300 may utilize a two- or three-channel nebulizer tube 401, 501 instead of the single-channel nebulizer tube 301. The inlet gas channel 405, 505 may be split downstream into two or three outlets 427, 527. Each of the outlet 527 may be fed by a separate liquid feed channel 506, FIG. 29. Experiments have shown that a multi-channel nebulizer tube configuration can decrease the time required to nebulize a given volume of liquid, thus minimizing the time needed to treat a patient.

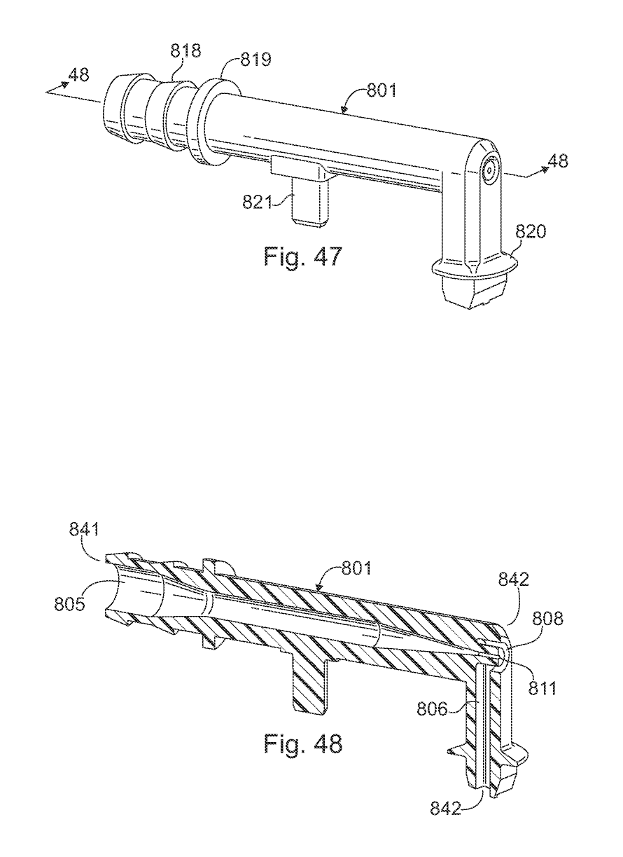

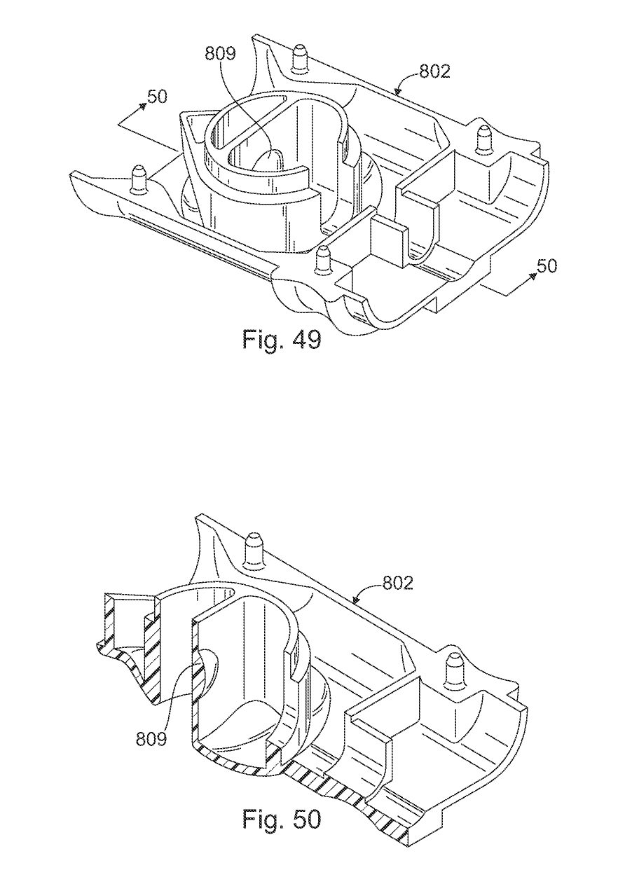

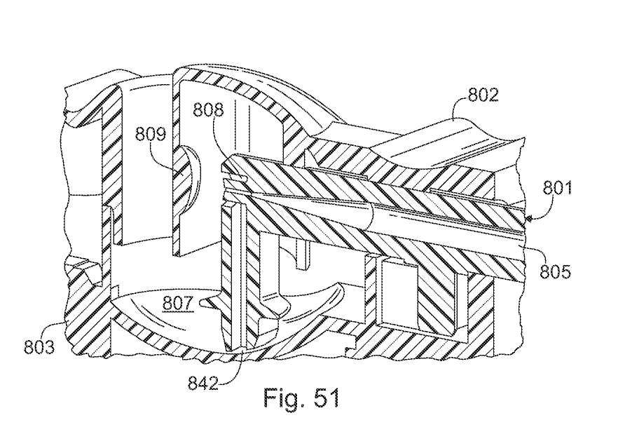

In addition, still further configurations of nebulizers and nebulizer tubes are provided by the present invention. For instance, with reference to FIGS. 47-48, a nebulizer tube 801 is provided, that may include similar structures to those of the nebulizer tube 1 of FIGS. 11-12, such as, an air baffle 20, an alignment boss 821, a barb 818, and a flange 819. In addition, the nebulizer tube 801 includes a gas channel 805 that may be provided in the form of a convergent channel 805 that has a cross-sectional dimension that decreases from the air inlet end 841 towards the opposing outlet end 842 which terminates at outlet nozzle 811. However, the nebulizer tube 801 includes an annular medication exit port 808 disposed in liquid communication with the liquid feed channel 806 through which liquid medication may be provided to the output end 842 of the nebulizer tube 801. The liquid feed channel 806 may have a generally rectangular or circular cross-sectional shape and have a cross-sectional dimension of 30-90 mils. The nebulizer tube 801 is disposed within the housing which may have upper and lower housing portions 802, 803 and which includes an impactor 809 proximate the outlet nozzle 811 and a reservoir 807 disposed in fluid communication with the feed channel 806, FIGS. 49-51.

In operation, a high pressure gas (typically air) enters the nebulizer tube 801 through the inlet end 841 and is accelerated to sonic velocity. The air expands as it leaves the nozzle 811. Since the feed channel 806 is in communication with a reservoir 811 of liquid (typically medication), under the proper conditions, liquid medication is siphoned through the feed channel 806 and exits the nebulizer tube 801 via annular medication exit port 808. Whether siphoning occurs depends on the spacing between the exterior face of the nozzle 811 and the impactor 809. Provided that the spacing between the exterior face of the nozzle 811 and the impactor 809 is sufficiently small (for example, 20 to 80 mils, with 30 mils representing a preferred spacing), a low-pressure air zone will be formed proximal to the annular medication exit port 808. This creates a pressure differential across the liquid that will siphon fluid from the reservoir 807 and direct it towards the impactor 809. The energy imparted to the liquid from the gas, as well as the impaction on the impactor 809, generates fine particles from the liquid.

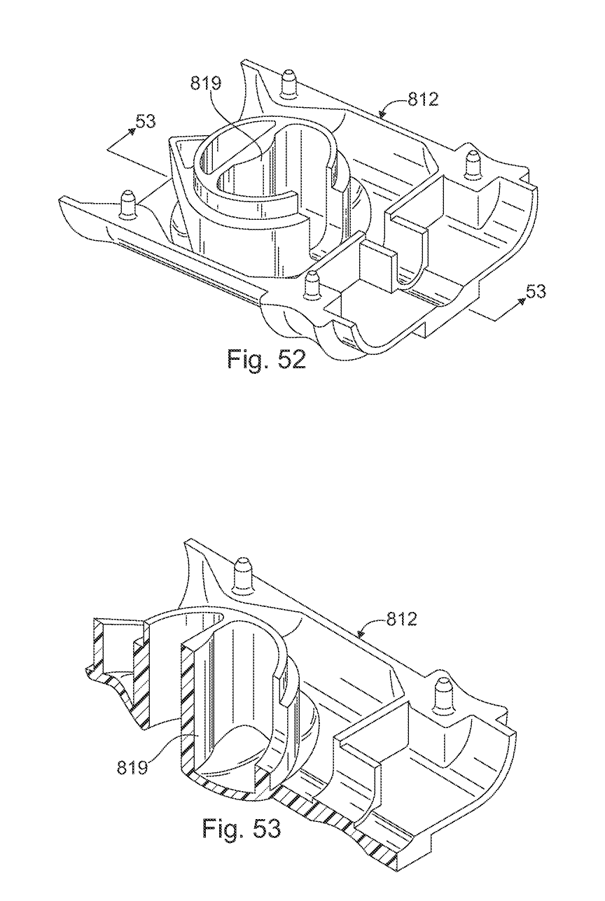

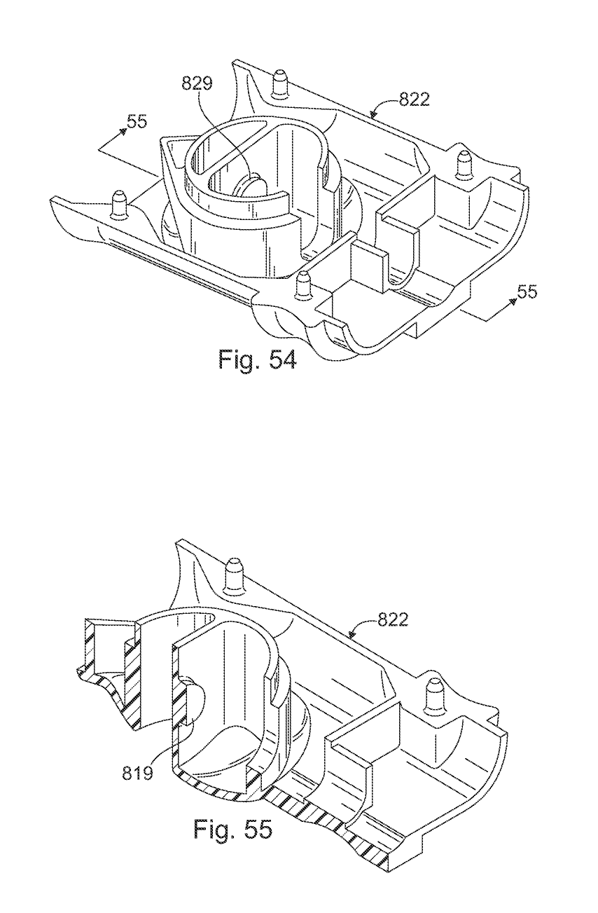

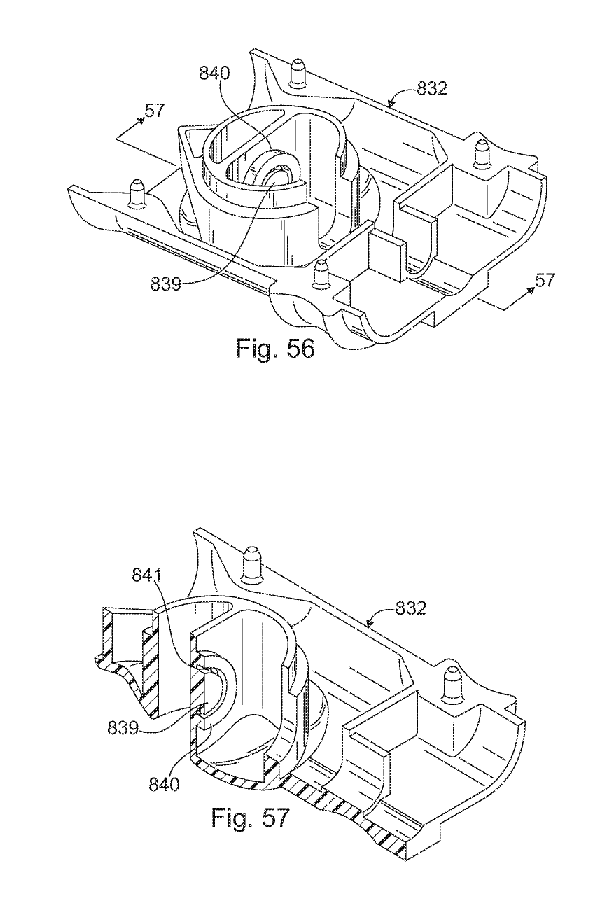

Further, alternative impactor structures in addition to the spherical impactor 809 of FIGS. 49-51 may be used in the present invention. For example, a cylindrical impactor 819 provided as part of an upper housing 812, FIGS. 52-53, or a mesa-shaped impactor 829 provided as part of an upper housing 822, FIGS. 54-55, may be used to increase the efficiency of the nebulization process. The mesa-shaped impactor 829 has been demonstrated to yield a relatively-high nebulization efficiency. It is believed that the turbulence that is generated as the air flow detaches from the circular edge of the mesa enhances efficiency. The flat surface of the mesa may be roughened to further enhance nebulization. Additionally, it is observed that the impaction surface of the mesa may be flat, convex, concave, or some other non-planar structure. The edges of the mesa may incorporate jagged features to further enhance efficiency. In addition, a ring feature 840 may be added about a mesa 839 to facilitate the creation of a resonant annular channel 841 in the housing 832, FIGS. 56-57. The fundamental resonant frequency of the annular channel 841 may be tuned to help generate particles of a preferred size. Also, the ring 840 can create more turbulence to increase efficiencies. Moreover, each of the impactor configurations illustrated in FIGS. 49-57 may be used with any of the other nebulizer and/or nebulizer tube configurations described herein.

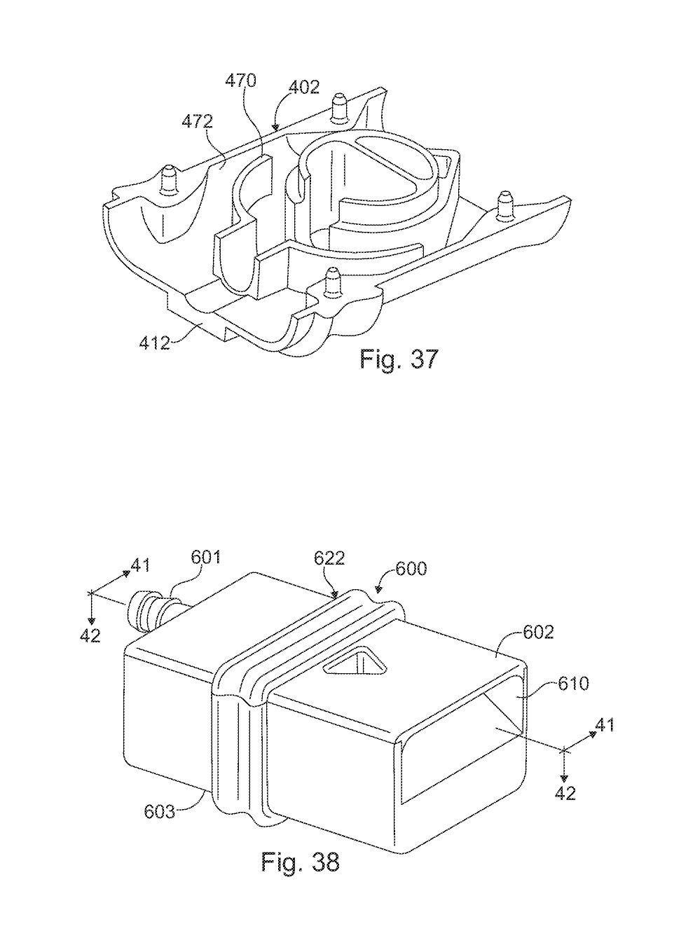

In yet another aspect of the present invention, a nebulizer configuration is provided in which the nebulizer body comprises only two parts, with the nebulizer tube 601 monolithically formed as a part of either the upper or the lower housing 602, 603, FIG. 38. Specifically, with reference to FIGS. 38-46 a nebulizer configuration in accordance with the present invention is shown in which the nebulizer tube 601 is formed as a part of the upper housing 602. As such, the nebulizer 600 may desirably include only two parts, the upper housing 602 and lower housing 603. However, as with the various nebulizer configurations 100, 200, 300 described above, one or more semi-permeable membranes (or filters) may additionally be provided at the exit port 610 to permit mist flow while acting as an effective liquid barrier to create a safety feature that prevents the user from swallowing liquid medication contained in the nebulizer 600. A sponge-like (or other absorbent) material may be incorporated into the nebulizer 600 as an alternative manner to obtain this feature. In the event that the nebulizer 600 is tilted beyond a critical angle during use, the membrane will block the flow of medication into the user's mouth.

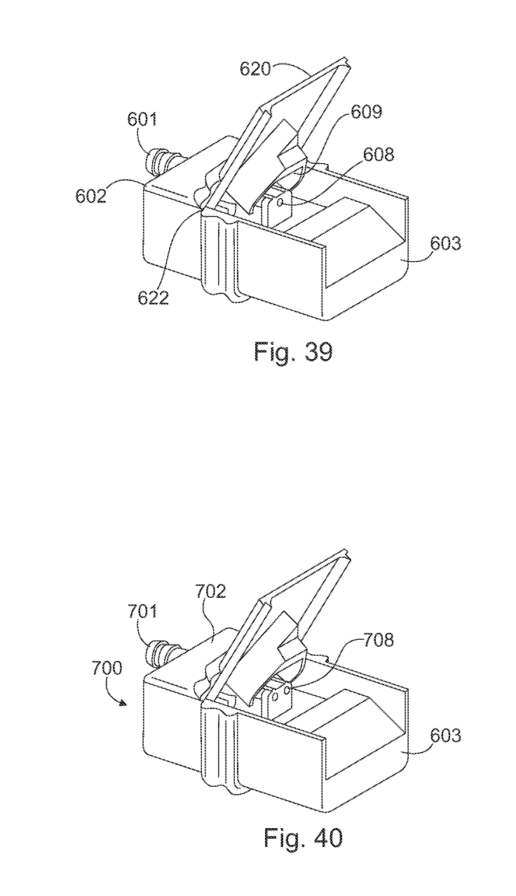

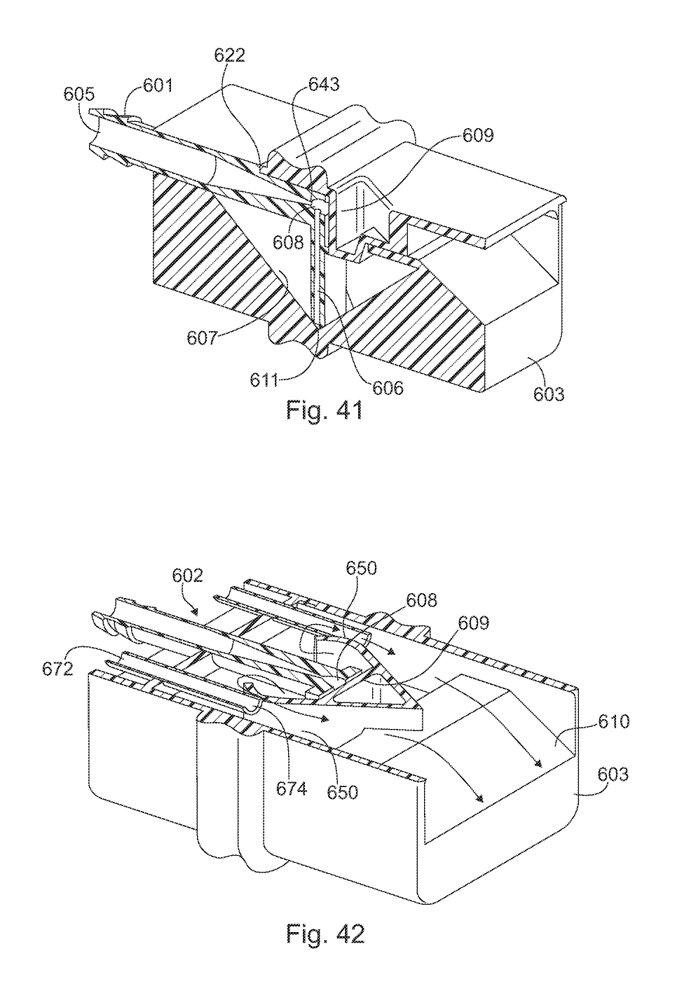

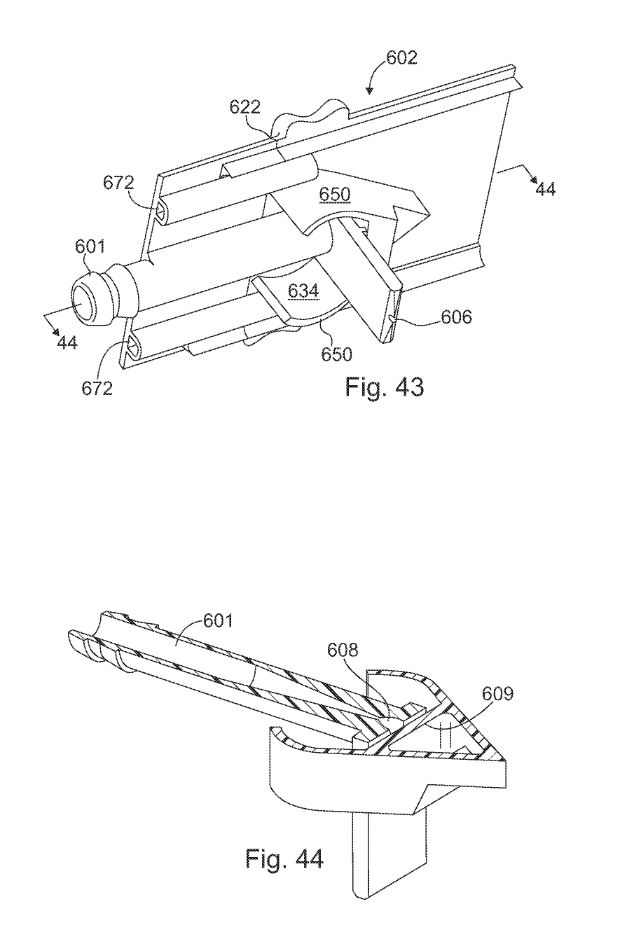

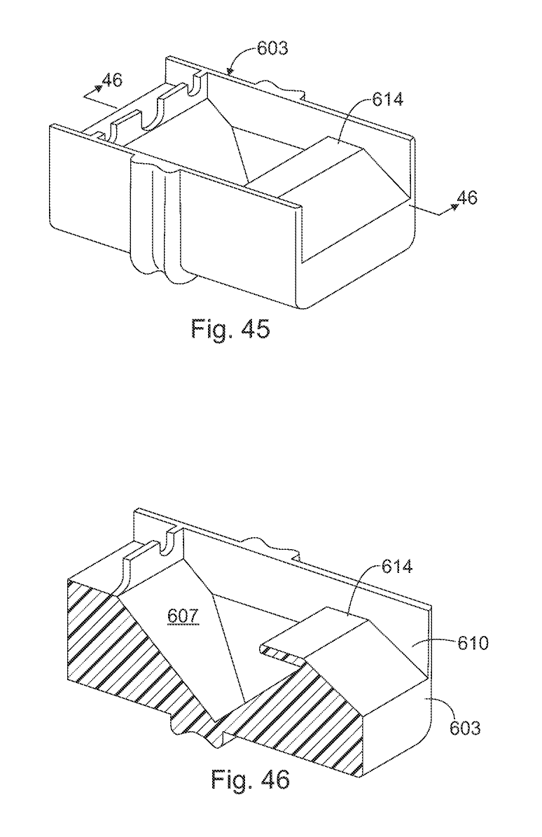

The upper housing 602 may include a “living hinge” 622 that allows the impactor half of the upper housing 602 to open as a lid 620 to permit the introduction of liquid medication into a reservoir 607 of the lower housing 603, FIG. 39. FIG. 43 shows the upper housing 602 after the living hinge 622 is flexed into its closed functioning orientation. To assist in maintaining the liquid medication in the reservoir 607, a medication retention flange 614 that extends over the reservoir 607 proximate the exit port 610 is provided as a part of the lower housing 603 to prevent medication from flowing out of the reservoir 607 and into the user's mouth, FIGS. 45, 46. The medication retention flange 614 allows the user to be inclined in bed or reclining while using this device. The reservoir 607 may be shaped to make the liquid medication available to the inlet end of the feed tube 606 of the nebulizer tube 601. For example, the reservoir 607 may be generally V-shaped and may include a trough 611 into which the liquid medication can pool and over which the inlet end of the feed tube 606 may be positioned to receive the pooled medication, FIG. 41. The lower end of the feed tube 606 may meet with the geometry of the reservoir 607 in the lower housing 603 such that medication in the reservoir 607 is wicked to the bottom of the feed tube 606 so that nearly all the medication can be siphoned into the air stream. (The feed tube 606 is the only feature that requires “side-action” to form the geometry.) The flat sloping walls that form the reservoir 607 allow the medication to be fully consumed even when the user is reclined at a significant angle.

The integral nebulizer tube 601 may also include a convergent channel 605 through which compressed air is introduced to the nebulizer 600. The air accelerates until it reaches the throat 643 (minimum cross-sectional area) of the tube. By virtue of the Bernoulli effect, as the flow velocity increases, its static pressure will decrease. As a result, the static pressure at the throat 643 of the nebulizer tube 601 is below that of the local atmospheric pressure. Since the static pressure of the liquid is higher than the static pressure at the throat 643 of the nebulizer tube 601, liquid is siphoned upward as a result of this Venturi effect. Subsequent to siphoning, the liquid/air mixture is rapidly expanded in the divergent section of a nozzle 608 of the nebulizer tube 601. This rapid expansion encourages turbulent mixing and creates an effective first-level of nebulization. After exiting the nozzle 608, the mixture strikes an impactor 609 which is also monolithic to the upper housing 602, FIGS. 41, 42, 44. This energetic collision generates the very fine, therapeutic particles required of nebulizers. Because the nozzle 608 and impactor 609 are both monolithic to the upper housing 602 through the living hinge 622, the spacing between the nozzle 608 and the impactor 609 is very repeatable, FIGS. 42, 44. A sufficiently small spacing between the nozzle 608 and the impactor 609 is required for fine mist generation, which may be, for example, about 30 thousandths of an inch.

As with the nebulizer configuration of FIG. 22, the nebulizer 600 may also include the “reverse flow” feature. Referring to FIG. 42, the reverse flow feature is illustrated where the mixture of air and nebulized medication (indicated by the lines with arrowheads), after hitting the impactor 609, is forced to flow back away from the exit port 610, then change direction to exit the nebulizer 600 at the exit port 610. In this regard, as with the nebulizer 300, the nebulizer 600 includes a nebulization chamber 634 defined and surrounded by chamber walls 650 that assist in defining the flow path of the mixture of air and nebulized medication. The change of flow direction acts as a filter, removing large droplets of medication from the air stream. The desired small airborne particles change direction with the air stream, while the larger particles with significantly more inertia do not readily change direction and impact the chamber walls or fall out of the air stream to head downward into the reservoir 607 to be reused.

In addition, the nebulizer 600 may also include one or more make-up air channels 672, which may be provided as a monolithic part of the upper housing 602, FIGS. 42, 43. The make-up air channels 672 allow the user to inhale or even exhale while using the nebulizer 600. The end 674 of the channel 672 inside the nebulizer 600 is positioned in the air stream such that the natural flow of the air stream will draw air into the nebulizer 600 rather than allowing nebulized medication to exit the make-up air channels 672 to the room. Furthermore, as with the various nebulizer configurations discussed above, the two-piece nebulizer 600 may make use of a nebulizer tube 701 that has more than one convergent/divergent gas channel and nozzle 708, FIG. 40. The inclusion of more than one gas channel can be achieved without significantly increasing the complexity of the mold tooling, which may be desirable because multiple gas channels can significantly reduce the time required to nebulize a certain amount of medication by drawing more medication through multiple feed tubes for mixing with high velocity air in multiple divergent nozzles 708.

The various nebulizer configurations presented above may have a compact size permitting the nebulizers to substantially fit within the user's mouth which contributes to minimizing the amount of residual medication. The compact size is not just a matter of design choice—it has an effect on all other aspects of the nebulizer's functionality. A higher nebulization rate, within a small volume, can have negative aspects. For example, there can be interaction between the multiple jets leading to an increased probability of particle agglomeration to a size larger than that desired for effective patient treatment. However, there can be substantial benefits of making the nebulizer very compact, such as high efficiency use of the medication, which is partially dependent upon having a compact nebulizer. A compact nebulizer has a smaller wettable surface area. Thus, the inner surfaces of the nebulizer will hold less residual medicine. The location and geometry of the liquid reservoir and intake, together with the gas flow path, are also important factors affecting the amount of residual. Thus, the designs strike a balance between nebulization rate and compactness.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A nebulizer for delivering a mist of liquid, comprising:
a housing;
a reservoir disposed internally to the housing for containing a liquid to be nebulized by the nebulizer;
a monolithic nebulizer tube having:
a gas channel having a first end for receiving a compressed gas and a second end for expelling compressed gas and nebulized liquid, the gas channel extending from a first end to a second end of the nebulizer tube, and
a liquid feed channel having a first end in fluid communication with the reservoir for receiving the liquid from the reservoir and having a second end in fluid communication with the gas channel, whereby application of compressed gas to the first end of the gas channel creates a siphon in the liquid feed channel to draw liquid into the feed channel and to expel the liquid and compressed gas from the second end of the nebulizer tube; and
a tortuous passageway disposed within the housing between second end of the gas channel and an exit port of the nebulizer for directing the flow of nebulized mist therethrough to the exit port.

2. The nebulizer according to claim 1, wherein the second end of the liquid feed channel communicates with the gas channel at a point intermediate the first and second ends of the gas channel.

3. The nebulizer according to claim 1, wherein the diameter of the gas channel has a minimum value at the point where the liquid feed channel communicates with the gas channel.

4. The nebulizer according to claim 1, wherein the gas channel comprises a nozzle disposed at the second end of the gas channel having a passageway that diverges towards the second end of the gas channel to enhance nebulization of the expelled liquid.

5. The nebulizer according to claim 1, wherein the nebulizer tube comprises a plurality of gas channels extending from the first end to the second end of the nebulizer tube and comprises a plurality of liquid feed channels, each feed channel in fluid communication with the reservoir and each feed channel comprising a second end in fluid communication with a respective one of the gas channels, whereby application of compressed gas to the first end of each gas channel creates a siphon in the respective liquid feed channel to draw liquid into each feed channel.

6. The nebulizer according to claim 1, wherein the tortuous passageway is configured to remove nebulized particles larger than a selected therapeutic size from the flow of nebulized mist.

7. The nebulizer according to claim 1, wherein the tortuous passageway comprises a first section for directing the flow of nebulized liquid away from the second end of the gas channel and back towards the inlet port of the housing.

8. The nebulizer according to claim 7, wherein the tortuous passageway comprises a second section for directing the flow of nebulized liquid to the exit port of the nebulizer.

9. A nebulizer for delivering a mist of liquid, comprising:
a housing;
a reservoir disposed internally to the housing for containing a liquid to be nebulized by the nebulizer; and
a monolithic nebulizer tube having:
a gas channel having a first end for receiving compressed gas and a second end for expelling compressed gas, the gas channel extending from a first end to a second end of the nebulizer tube, and
a liquid feed channel having a first end in fluid communication with the reservoir for receiving the liquid from the reservoir and having an annular passageway at a second end of the feed channel, the annular passageway disposed about the second gas channel end, whereby application of compressed gas to the first end of the gas channel creates a siphon in the liquid feed channel to draw liquid into the feed channel and to expel the liquid and compressed gas from the second end of the feed channel.

10. The nebulizer according to claim 1 or 9, wherein the gas channel tapers in diameter from a relatively larger diameter at the first end of the gas channel to a relatively smaller diameter between the first and second gas channel ends.

11. The nebulizer according to claim 1 or 9, wherein the nebulizer tube is a monolithic part of the housing.

12. The nebulizer according to claim 11, wherein the housing consists of only two pieces and the reservoir is monolithic to the housing.

13. The nebulizer according to claim 1 or 9, wherein the housing consists of only two pieces and the reservoir is monolithic to the housing.

14. The nebulizer according to claim 1 or 9, comprising an impactor disposed proximate the second end of the gas channel to nebulize the expelled liquid when the expelled liquid strikes the impactor.

15. The nebulizer according to claim 14, wherein the impactor comprises a spherical or cylindrical shape.

16. The nebulizer according to claim 14, wherein the impactor comprises a mesa.

17. The nebulizer according to claim 14, wherein the impactor comprises a mesa with a ring disposed around the mesa to provide an annular channel between the ring and the mesa.

18. The nebulizer according to claim 17, wherein the annular channel is dimensioned to provide a fundamental resonant frequency of the annular channel tuned to generate particles of a preferred size.

19. The nebulizer according to claim 14, wherein the impactor is disposed sufficiently close the second end of the gas channel to assist siphoning the liquid into the feed channel.

20. The nebulizer according to claim 14, wherein the impactor is disposed sufficiently close the second end of the gas channel to assist in nebulizing the liquid expelled from the second end of the gas channel.

21. The nebulizer according to claim 20, wherein the impactor is disposed 15 to 30 thousandths of an inch away from the second end of the gas channel.

22. The nebulizer according to claim 14, wherein the impactor has a curved surface that curves away from the second end of the gas channel to assist in directing the nebulized liquid towards an exit port of the nebulizer for inhalation by a user.

23. The nebulizer according to claim 22, wherein the impactor has an airfoil cross-sectional shape that is oriented in the housing to direct nebulized liquid towards the exit port of the nebulizer to reduce turbulence in the exiting stream of nebulized liquid and to reduce backpressure at the exit port.

24. The nebulizer according to claim 23, wherein the airfoil includes a tapered portion pointing in a downstream direction towards the exit port of the nebulizer.

25. The nebulizer according to claim 14, wherein the impactor is disposed above the reservoir such that droplets of liquid that collect on the impactor are drawn by gravity downward into the reservoir to be recycled into the liquid feed channel.

26. The nebulizer according to claim 1 or 9, comprising a nebulization chamber disposed within the housing with the second end of the gas channel disposed internal to the nebulization chamber, the nebulization chamber disposed above the reservoir at a location such that droplets of liquid that collect on a surface of the nebulization chamber are drawn by gravity downward into the reservoir to be recycled into the liquid feed channel.

27. The nebulizer according to claim 1 or 9, comprising a tortuous passageway disposed between the second end of the gas channel and an exit port of the nebulizer for directing the flow of nebulized mist therethrough to the exit port.

28. The nebulizer according to claim 27, wherein the tortuous passageway is configured to remove nebulized particles larger than a selected therapeutic size from the flow of nebulized mist.

29. The nebulizer according to claim 27, wherein the tortuous passageway comprises a first section for directing the flow of nebulized liquid away from the second end of the gas channel and back towards the first end of the gas channel.

30. The nebulizer according to claim 29, wherein the tortuous passageway comprises a second section for directing the flow of nebulized liquid to an exit port of the nebulizer.

31. The nebulizer according to claim 1, wherein the nebulizer tube comprises an air baffle disposed proximate the first end of the liquid feed channel to deter liquid being blown away from the first end of the liquid feed channel by the air flow from the second end of the gas channel.

32. The nebulizer according to claim 1 or 9, wherein the nebulizer tube comprises an alignment boss for registration with a complementary alignment feature of the housing to position the nebulizer tube at a selected location within the housing.

33. The nebulizer according to claim 1 or 9, wherein the reservoir comprises a sloping wall to direct the liquid towards the second end of the liquid feed channel to enhance full consumption of the liquid.

34. The nebulizer according to claim 33, wherein the reservoir is substantially V-shaped.

35. The nebulizer according to claim 33, wherein the sloping wall is oriented so that liquid is collected at the location of the first end of the liquid feed channel even when the nebulizer is tilted at a significant angle.

36. The nebulizer according to claim 1 or 9, wherein the reservoir comprises a sidewall having grooves oriented in a direction down the sidewall to direct liquid down the sidewall to the first end of the liquid feed channel.

37. The nebulizer according to claim 1 or 9, comprising a gap between the first end of the liquid feed channel and a bottom surface of the reservoir that is sufficiently small to cause liquid to wick into the gap.

38. The nebulizer according to claim 37, wherein the gap that becomes smaller as it approaches the inlet to the feed channel to encourage the liquid to flow in the direction of the inlet to the feed channel.

39. The nebulizer according to claim 1 or 9, comprising a membrane disposed at an exit port of the nebulizer, the membrane structured to allow mist flow therethrough and deterring the passage of liquid therethrough.

40. The nebulizer according to claim 1 or 9, comprising an absorbent material proximate an exit port of the nebulizer to capture liquid that may spill from the reservoir to deter the passage of liquid through the exit port.

41. The nebulizer according to claim 1 or 9, comprising an overflow wall disposed between the reservoir and an exit port of the nebulizer to deter liquid spillage through the exit port when the nebulizer is tilted downward towards the exit port.

42. The nebulizer according to claim 41, wherein the overflow wall extends over the reservoir to overhang the reservoir.

43. The nebulizer according to claim 1 or 9, comprising a fill port for receiving liquid for delivery to the reservoir, the fill port configured to permit the nebulized liquid to exit therethrough.

44. The nebulizer according to claim 1 or 9, comprising a fill port for receiving liquid for delivery to the reservoir and comprising a fill port cap monolithic to the housing.

45. The nebulizer according to claim 1 or 9, comprising a fill port for receiving liquid for delivery to the reservoir and comprising a fill port cap disposed internally to the housing deter the cap from coming loose during use.

46. The nebulizer according to claim 1 or 9, comprising a fill port for receiving liquid for delivery to the reservoir and comprising a fill port cap configured to deflect upon application of a suitable force to permit introduction of liquid through the cap into the reservoir.

47. The nebulizer according to claim 1 or 9, comprising a fill port for receiving liquid for delivery to the reservoir, and comprising a fill port cap having a one-way valve to permit the flow of liquid therethrough and to deter the flow of nebulized mist therethrough.

48. The nebulizer according to claim 1 or 9, comprising a fill port having a geometry to accept the shape of standard medicine containers to facilitate filling of the nebulizer with the selected amount of liquid and reduce the possibility of spillage and waste.

49. The nebulizer according to claim 1 or 9, comprising a make-up air passageway extending from an inlet end of the nebulizer to an exit port of the nebulizer to permit air to be drawn by the user from the inlet end through to the nebulizer to the exit port.

50. The nebulizer according to claim 1 or 9, wherein the housing comprises a wetting material to create a sheeting action for facilitating the flow of liquid deposited on the interior surfaces the housing into the reservoir.

51. A nebulizer for delivering a mist of liquid, comprising:
a housing having an inlet port for receiving compressed gas and an exit port for delivering a mist of nebulized liquid;
a reservoir disposed internally to the housing for containing a liquid to be nebulized by the nebulizer;
a nebulizer tube in fluid communication with the liquid having an outlet from which the nebulized mist is provided, the outlet end disposed internally to the housing; and
a tortuous passageway disposed within the housing between outlet end of the nebulizer tube and the exit port of the nebulizer for directing the flow of nebulized mist therethrough to the exit port.

52. The nebulizer according to claim 51, wherein the tortuous passageway is configured to remove nebulized particles larger than a selected therapeutic size from the flow of nebulized mist.

53. The nebulizer according to claim 51, wherein the tortuous passageway comprises a first section for directing the flow of nebulized liquid away from outlet end of the nebulizer tube and back towards the inlet port of the housing.

54. The nebulizer according to claim 53, wherein the tortuous passageway comprises a second section for directing the flow of nebulized liquid to the exit port of the nebulizer.

55. The nebulizer according to claim 51, comprising an impactor disposed proximate the outlet end of the nebulizer tube to nebulize the expelled liquid when the expelled liquid strikes the impactor.

56. The nebulizer according to claim 55, wherein the impactor comprises a spherical or cylindrical shape.

57. The nebulizer according to claim 55, wherein the impactor comprises a mesa.

58. The nebulizer according to claim 55, wherein the impactor comprises a mesa with a ring disposed around the mesa to provide an annular channel between the ring and the mesa.

59. The nebulizer according to claim 58, wherein the annular channel is dimensioned to provide a fundamental resonant frequency of the annular channel tuned to generate particles of a preferred size.

60. The nebulizer according to claim 55, wherein the impactor is disposed sufficiently close the outlet end of the nebulizer tube to assist siphoning the liquid into the nebulizer tube.

61. The nebulizer according to claim 55, wherein the impactor is disposed sufficiently close the second end of the gas channel to assist in nebulizing the liquid expelled from the outlet end of the nebulizer tube.

62. The nebulizer according to claim 61, wherein the impactor is disposed 15 to 30 thousandths of an inch away from the outlet end of the nebulizer tube.

63. A two-piece nebulizer for delivering a mist of liquid, comprising:
a two-piece housing having separate first and second housing portions;
a reservoir monolithic to the housing for containing liquid to be nebulized by the nebulizer; and
a nebulizer tube monolithic to the housing having:
a gas channel having a first end for receiving compressed gas and a second end for expelling compressed gas and nebulized liquid, the gas channel extending from a first end to a second end of the nebulizer tube,
a liquid feed channel having a first end in fluid communication with the reservoir for receiving liquid from the reservoir and having a second end in fluid communication with the gas channel, whereby application of compressed gas to the first end of the gas channel creates a siphon in the liquid feed channel to draw liquid into the feed channel and to expel the liquid along with compressed gas from the second end of the nebulizer tube.

64. The nebulizer according to claim 63, wherein the housing comprises an impactor monolithic to the housing and disposed proximate the second end of the gas channel to nebulize the expelled liquid when the expelled liquid strikes the impactor.

65. The nebulizer according to claim 63, wherein the impactor comprises a spherical or cylindrical shape.

66. The nebulizer according to claim 63, wherein the impactor comprises a mesa.

67. The nebulizer according to claim 63, wherein the impactor comprises a mesa with a ring disposed around the mesa to provide an annular channel between the ring and the mesa.

68. The nebulizer according to claim 67, wherein the annular channel is dimensioned to provide a fundamental resonant frequency of the annular channel tuned to generate particles of a preferred size.

69. The nebulizer according to claim 63, wherein the reservoir is substantially V-shaped.
